(19) United States
(12) Patent Application Publication
(10) Pub. No.: US 2014/0134140 A1
(43) Pub. Date: May 15, 2014

(54) MESENCHYMAL STEM CELL COMPOSITIONS FOR THE TREATMENT OF MICROBIAL INFECTIONS

(71) Applicant: Case Western Reserve University, Cleveland, OH (US)

(72) Inventors: Arnold I. Caplan, Cleveland Heights, OH (US); Tracey L. Bonfield, Chesterland, OH (US)

(21) Appl. No.: 14/077,946

(22) Filed: Nov. 12, 2013

Related U.S. Application Data
(60) Provisional application No. 61/724,555, filed on Nov. 9, 2012.

(51) Int. Cl. A61K 35/28

(52) U.S. Cl. CPC A61K 35/28
USPC 424/93.7

(57) ABSTRACT

A method of treating a microbial infection in a subject includes administering to the subject a therapeutically effective amount of MSCs and an antimicrobial agent, wherein the MSCs potentiate the therapeutic activity of the antimicrobial agent, and the therapeutically effective amount is the amount effective to inhibit microbial growth and suppress microbial infection associated inflammation in the subject.

### RELATED APPLICATION

This application claims priority from U.S. Provisional Application No. 61/724,555, filed Nov. 9, 2012, the subject matter of which is incorporated herein by reference in its entirety.

### GOVERNMENT FUNDING

This invention was made with government support under Grant No's P30, DK27651-29, R21 HL109699, R21 HL114268, and R21 HL104362, awarded by The National Institutes of Health. The United States government may have certain rights to the invention.

### TECHNICAL FIELD

This application relates to antimicrobial compositions and methods for treating microbial infections and more particularly to compositions and methods for treating respiratory microbial infections associated with Cystic Fibrosis.

### BACKGROUND

Microbial infections are a significant cause of morbidity and mortality globally. Resistance to existing antimicrobial therapies coupled with the decline in the development of new alternatives necessitates the search for compositions that can prevent and treat serious microbial infections.

The microbes Pseudomonas aeruginosa, Staphylococcus aureus and Streptococcus pneumoniae are major contributors to infections associated with community acquired and nosocomial bacteremia in humans. Illnesses associated with these pathogens range from minor skin infections to life-threatening diseases such as pneumonia, meningitis, toxic shock syndrome and sepsis. Current empiric treatment primarily consists of antibiotics. As the body utilizes the available antibiotics, there is a depleted efficiency in combating bacterial infections. Efficiency in utilization of antibiotics, poor compliance in taking the medicine, and frequent use can result in the development of antibiotic resistant bacteria. The loss of antibiotic effectiveness can lead to progression into bacteremia and sepsis. Side effects of antibiotic usage have also been identified as well as correlations between antibiotic use and cancer, allergic reactions, destruction of the beneficial bowel flora leading to mal-adsorption syndromes and food allergies, development of resistant species, immune suppression, chronic fatigue syndrome, and nutrition deficiency. These side effects are in addition to the economical cost of antibiotic therapy, and the cost of hospitalization. Therefore, there remains a medical need for novel compositions and methods effective in the treatment of microbial infections, while preventing the development of resistant strains and antimicrobial toxicity.

### SUMMARY

Embodiments described herein relate generally to anti-microbial compositions and methods for treating microbial infections and more particularly to compositions and methods for respiratory microbial infections associated with cystic fibrosis.

A first aspect of the application relates to a method of treating a microbial infection in a subject. The method includes administering to the subject a therapeutically effective amount of mesenchymal stem cells (MSCs), wherein the therapeutically effective amount is the amount effective to inhibit microbial growth and suppress microbial infection associated inflammation in the subject.

Another aspect of the application relates to a method of treating a microbial infection in a subject. The method includes administering to the subject a therapeutically effective amount of mesenchymal stem cells (MSCs) and an antimicrobial agent, wherein the MSCs potentiate the therapeutic activity of the antimicrobial agent, and wherein the therapeutically effective amount is the amount effective to inhibit microbial growth and suppress microbial infection associated inflammation in the subject.

In yet another aspect of the invention relates to a method of treating a respiratory microbial infection associated with cystic fibrosis in a subject. The method includes administering to the subject a therapeutically effective amount of MSCs and an antimicrobial agent, wherein the MSCs potentiate the therapeutic activity of the antimicrobial agent, and wherein the therapeutically effective amount is the amount effective to inhibit microbial growth and suppress respiratory microbial infection associated inflammation in the subject.

A further aspect of the application relates to an antimicrobial pharmaceutical composition. The antimicrobial pharmaceutical composition includes an effective amount of MSCs and an antimicrobial agent, wherein the effective amount of MSCs potentiate the therapeutic activity of the antimicrobial agent.

### BRIEF DESCRIPTION OF THE DRAWINGS

FIG. 1 illustrates that hMSCs attenuate weight loss in the Chronic Infection/Inflammation Murine Model of CF. In each of the experiments (n=3 different hMSC or BMM preparations), Cftr^{tm2Uth} and wild type littermate controls (5-8 animals/group/study at 16-24 animals total per experiment) were infected with 5×10⁵ CFUs of Pseudomonas aeruginosa (PA M5715) impregnated into agar beads. 24 hours post-infection, half of the mice were given 1×10⁶ hMSCs or BMM through the retro-orbital sinus. Animals were followed for either 3 or 10 days for weight loss. Treatment of WT and Cftr^{tm2Uth} mice with hMSCs attenuated weight loss, with the Cftr^{tm2Uth} mice reaching significance at both day 3 and day 10 (P≦0.05). Further, BMM treatment resulted in a statistical increase in weight (P≦0.05) relative to the non-treated control at day 10. We used the nonparametric Mann-Whitney t-test for the paired comparisons and the one-way ANOVA for the multiple comparisons.

FIG. 2 illustrates hMSCs decrease clinical score associated with chronic Pseudomonas aeruginosa infection. Animals described in FIG. 1 were followed for 10 days for clinical score (Table 1) and then euthanized. Data is expressed as cumulative clinical score at day 3 (FIG. 2A) and day 10 (FIG. 2B). Cftr^{tm2Uth} mice had elevated clinical scores relative to WT mice at day 3, it did not reach significance. Cftrtm2Uth mice did have elevated clinical score at day 10 relative to WT mice infected at the same time (P≦0.05, paired t-test). Administration of hMSCs and BMM resulted in a trend toward clinical improvement day 3 (FIG. 2A, P=0.07) and reached significance at day 10 (FIG. 2B, P≦0.05) using one-way ANOVA analyses.

FIG. 3 illustrates that hMSCs decrease lung pathology associated with chronic Pseudomonas aeruginosa infection. Animals described in FIGS. 1 and 2 were also evaluated for lung pathology using the criteria outlined in Table 1. Cftr^{tm2Uth} mice tended to have higher gross lung pathology scores compared to WT mice (p=0.07). Treatment of Cftr^{tm2Uth} mice with hMSCs resulted in significantly improved gross lung pathology (P≦0.05). BMM had no statistical impact on gross lung pathology. We used the non-parametric Mann Whitney t-test for these analyses.

FIG. 4 illustrates hMSC and cell recruitment into the lungs. Animals were euthanized and BAL was obtained for absolute white cell counts. Cftr^{tm2Uth} mice had elevated levels of leukocyte recruitment relative to WT mice (P=0.05) which was significantly decreased to levels approaching WT mice with the administration of either hMSCs or BMM (P≦0.05). We used the non-parametric Mann-Whitney t-test for these analyses.

FIG. 5 illustrates that hMSCs decrease neutrophils while increasing alveolar macrophages. The BAL obtained from the in vivo studies was also evaluated for cellular differential. There was no difference in lymphocyte or eosinophil counts between the WT and Cftr^{tm2Uth} with or without hMSC or BMM administration (data not shown). A Cftr^{tm2Uth} had elevated neutrophils (5B, P≦0.05) and decreased alveolar macrophages (5A, P≦0.05) relative to WT controls infected at the same time. Treatment of Cftr^{tm2Uth} mice with either hMSCs or BMM, resulted in increased alveolar macrophages (5A, P≦0.05), while decreasing neutrophil numbers (5B, P≦0.05). We used the non-parametric Mann-Whitney t-test for the paired comparisons and the one-way ANOVA for the multiple comparisons.

FIG. 6 illustrates hMSC supernatants enhance macrophage recruitment. Alveolar macrophages and peritoneal neutrophils were obtained from WT and Cftr^{tm2Uth} mice and cultured in transwells (0.4 μm) against three different batches of MSC supernatants compared to controls (WT or Cftr^{tm2Uth} cells with medium alone). Cellular recruitment was measured by counting the cells in the lower chamber after 4 hours incubation at 370° C. There was no difference between WT and Cftr^{tm2Uth} cells cultured with control medium (only WT data is shown). Supernatant derived from hMSCs significantly recruitment Cftr^{tm2Uth} alveolar macrophages relative to WT cells and controls (P≦0.05). Due to un-equal variance the Wilcoxon signed ranked test was used for these analyses.

FIG. 7 illustrates that hMSCs shift cytokines away from neutrophil recruitment and Pro-inflammation. Cftr^{tm2Uth} mice had elevated KC (7A), IL-6 (7B), IL-1B (7C) MIP-2 (D), adiponectin (E) and resistin (F) concentrations relative to WT controls when comparing of variance the mean. hMSC and BMM administration decreased KC, IL-6 and IL-1B (P≦0.05, P=0.07) with BMM giving a more dramatic effect (P≦0.05). BMM also decreased adiponectin and MIP-2 (P≦0.05). We used the non-parametric Mann-Whitney t-test for the paired comparisons and the one-way ANOVA for the multiple comparisons.

FIG. 8 illustrates the mechanisms of anti-inflammation. Using in vitro modeling with BMM from Cftr^{tm2Uth} and WT mice we measured changes in LPS induced IL-6 (8A) and TNFα (8B) when cultured in the presence or absence of hMSC derived supernatants. Cftr^{tm2Uth} expressed greater levels of both IL-6 and TNFα mRNA post-stimulation with LPS (FIGS. 8A and 8B respectively, P≦0.05, n=3 different hMSC preparations). WT bone marrow cells expressed comparable levels of IL-6 and TNFα mRNA regardless of co-culture conditions (with or without hMSC supernatants). In panels C and D, transformed human tracheal epithelial cells from a CF patient (IB3) and the corrected control HC (S9) were also cultured with LPS in the presence and absence of hMSC supernatant and evaluated for IL-8 (8C) and IL-6 (8D) mRNA. LPS significantly induced epithelial IL-8 (P≦0.05) and IL-6 (P≦0.05) mRNA expression relative to the controls Like the BMM derived cells, supernatants derived from hMSCs significantly decreased IL-6 and IL-8 mRNA synthesis in response to exposure to LPS (P≦0.05, n=3 different hMSC preparations). The students t-test was used for these analyses.

FIG. 9 illustrates hMSC bactericidal activity. Whole lung homogenates and BAL fluid were obtained from the in vivo models and evaluated for bacterial load (colony forming units). Both WT and Cftr^{tm2Uth} mice had elevated and comparable CFUs post-Infection at day 3. The level of CFUs were significantly decreased by the administration of both hMSCs and WT BMM (9A, P≦0.05, for n=3 different hMSC and BMM preparations). To determine if this decrease in bacterial load was due to hMSC products or host response, hMSC supernatants were cultured with Pseudomonas aeruginosa (10⁴ CFU). Supernatants were used from un stimulated hMSCs (cultured in plastic) or hMSCs stimulated for 24 hours with LPS (0.5 μg/ml). Supernatants from the hMSCs significantly decreased bacterial CFUs (9B, n=3 different hMSC derived supernatants, P≦0.05). We used the non-parametric Mann-Whitney t-test for these analyses.

FIG. 10 illustrates that Cftr^{tm2Uth} and WT mice BAL have elevated LL-37 post-hMSCs administration. Cftr^{tm2Uth} and WT mice were chronically infected with Pseudomonas aeruginosa with and without either hMSC or BMM therapy and followed for 10 days. Animals were euthanized with BAL. BAL was evaluated for LL-37 using an ELISA based methodology. Cftr^{tm2Uth} mice and WT mice had comparable levels of LL-37, administration of hMSCs but not BMM increased LL-37 (n=3 different studies; 4-6 samples/group, P≦0.05). We used the F-test to compare variances between Cftr^{tm2Uth} with and without MSCs along with like treated controls. The Mann-Whitney t-test value was P=0.07.

FIG. 11 illustrates that hMSCs and BMM cells secrete LL-37. hMSCs utilized in the animal models were cultured in vitro with and without the addition of 0.5 ug/ml LPS (n=5 preparations) with or without inhibition of CFTR with I-172 (10 ug/ml for 48 hours) and incubated for 24 hours. The supernatants were harvested and evaluated for LL-37 concentration. hMSCs secreted LL-37 with and without LPS stimulation. Inhibition of CFTR function significantly inhibited LL-37 secretion by hMSCs with or without LPS stimulation (P≦0.05). We used the student t-test for these analyses.

FIGS. 12(A-C) are graphs showing MSC-supernatants enhanced overall efficiency of the antibiotic geneticin (100 μg/ml, n=3, P≦0.05). MSC-supernatants were cultured with 10⁴ CFU of Pseudomonas aeruginosa (12A) Staphylococcus aureus (12B) or Streptococcus pneumonia (12C). MSC-supernatants decreased Pseudomonas aeurginosa (n=3), Staphylococcus aureus (n=3) and Streptcoccus pneumoniae (n=2) growth.

FIG. 13 is a graph that shows MSC derived products and antimicrobial activities. MSC-supernatants enhanced overall efficiency of the antibiotic geneticin (100 μg/ml, n=3, P≦0.05) through changing the proliferative capacity of the bacteria.

FIG. 14 is a schematic illustration of Ex Vivo antimicrobial studies. Tracheal aspirates from patients are cultured with and without MSCs or their secreted products and evaluated for growth differences.

FIG. 15 illustrates MRI images tracking infection/inflammation in mice. Mice were given by tail vein injection a slurry of perfluorocarbon-conjugated particles the day after instillation of agarose beads, with (+PA) and without Pseudomonas (no PA). The particles are phagocytosed and tracked by MRI of fluorine (shown in red), overlaid on the hydrogen signal in black and white. The arrow points out the signal in the lungs of the infected non-CF mice.

FIG. 16 illustrates the efficiency of luciferase and red fluorescent protein labeling of BMD-hMSCs and imaging. hMSCs were cultured with luciferase and red fluorescent protein prior to administration to mice which had their right leg irradiated to monitor cellular recruitment to areas of injury. At day 2, the bone marrow cells dispersed in the lung, liver and damaged leg remain visible. By week 1, the focus is liver and damaged leg. By week 3, the hMSCs are localized to the irradiated leg only.

FIG. 17 is a graph showing the bactericidal activity of MSC-cells in the Pseudomonas aeruginosa Pneumonia Model. Whole lung homogenates and BAL fluid were obtained from the in vivo model of gram negative Pseudomonas aeruginosa and evaluated for bacterial load. There was significant CFUs post-Infection. The level of CFUs were significantly decreased by the administration of MSCs (3A, P≦0.05, for n=3).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Lodish et al., Molecular Cell Biology, 6th Edition, W. H. Freeman: New York, 2007, and Lewin, Genes IX, Jones and Bartlett Publishers: Mass., 2008. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The term “treatment” or “treating” refers to any therapeutic intervention in a mammal, including: (i) prevention, that is, causing the clinical symptoms not to develop, e.g., preventing infection from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection.

The terms “reducing”, “suppressing” and “inhibiting” have their commonly understood meaning of lessening or decreasing.

The terms “effective,” “effective amount,” and “therapeutically effective amount” refer to that amount of MSCs, compositions including both MSCs and an antimicrobial agent, and/or a pharmaceutical composition thereof that inhibits the growth of one or more microbes in a subject and/or that results in amelioration of symptoms (e.g., infection induced inflammation) or a prolongation of survival in a subject with a microbial related disease or disorder.

The term “growth” as used herein refers to a growth of one or more microorganisms and includes reproduction or population expansion of the microorganism (e.g. bacteria). The term also includes maintenance of on-going metabolic processes of a microorganism, including processes that keep the microorganism alive.

The phrases “parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “pharmaceutical composition” refers to a formulation containing the therapeutically active agents described herein in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., MSCs and antimicrobial agents described herein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. In a preferred embodiment, the active ingredients are mixed under sterile conditions with a pharmaceutically acceptable carrier, and with any preservatives, buffers, or propellants that are required.

The terms “pharmaceutically acceptable” or “therapeutically acceptable” refers to a substance which does not interfere with the effectiveness or the biological activity of the active ingredients and which is not toxic to the host.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder (e.g., a microbial infection).

The term “in vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

The term “antimicrobial agent” refers to any molecule or other agent that is a biologically, physiologically, or pharmacologically active substance that acts locally or systemically in a subject to substantially reduce or inhibit microbial growth, for example, a antibacterial agent capable of reducing the proliferative capacity of bacteria. In some embodiments, an antimicrobial agent can include an antibiotic, antifungal, antiviral, and/or antiparasitic agent.

The term “antibiotic agent” as used herein refers to any substance, compound or a combination of substances or a combination compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibiotic” also refers to compounds capable of decreasing infectivity or virulence of bacteria.

The term “beta(β)-lactam antibiotic” as used herein refers to compounds with antibiotic properties and containing a beta-lactam nucleus in their molecular structure.

The term “antifungal agent” as used herein refers to any substance, compound or a combination of substances or a combination compounds capable of: (i) inhibiting, reducing or preventing growth of fungi; (ii) inhibiting or reducing ability of fungi to produce infection in a subject; or (iii) inhibiting or reducing ability of fungi to multiply or remain infective in the environment.

The term “antiviral drug” or “antiviral agent” as used herein refers to any substance, compound or a combination of substances or a combination compounds capable of: (i) inhibiting, reducing or preventing growth of a virus; (ii) inhibiting or reducing ability of a virus to produce infection in a subject; or (iii) inhibiting or reducing ability of virus to replicate or remain infective in the environment. Like antibiotics for bacteria, specific antivirals are typically used for specific viruses.

The term “Cystic fibrosis (CF)” refers to an autosomal recessive disorder with a highly variable clinical presentation. Cystic fibrosis is predominantly a disorder of infants, children and young adults, in which there is widespread dysfunction of the exocrine glands, characterized by signs of chronic pulmonary disease, pancreatic deficiency, abnormally high levels of electrolytes in the sweat and occasionally by biliary cirrhosis. Also associated with the disorder is an ineffective immunologic defense against microbes as well as dysregulated inflammation in the lungs. The classic form of cystic fibrosis is caused by loss-of-function mutations in the cystic fibrosis transmembrane conductance regulator (CFTR) gene. Non-classic forms of cystic fibrosis have been associated with mutations that reduce but do not eliminate the function of the CFTR protein.

As used herein, the terms “subject suffering from cystic fibrosis”, “subject having cystic fibrosis” or “subjects identified with cystic fibrosis” refers to subjects that are identified as having or likely having a mutation in the gene that encodes cystic fibrosis transmembrane conductance regulator (CFTR) protein, which cause cystic fibrosis. For the purposes of this application, the terms “cystic fibrosis-related disease(s) or disorder(s)” includes diseases and/or conditions related to Cystic Fibrosis (CF). Examples of such diseases include cystic fibrosis, variant cystic fibrosis and non-CF bronchiectasis.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to the use of MSCs alone or in combination with antimicrobial agents and methods for treating microbial infections and more particularly to compositions and methods for treating respiratory microbial infections in subjects having cystic fibrosis.

It has been shown that MSCs are environmentally responsive and have the capacity to secrete factors that are both anti-inflammatory and anti-microbial, thus attenuating inflammation while at the same time aiding in infection resolution associated with microbial infection. For example, FIGS. 1-3 of the application show for the first time in an in vivo model mimicking lung infection and inflammation in cystic fibrosis, that retro-orbital administration of MSCs resulted in attenuated weight loss, alone with decreased clinical score and lung pathology associated with chronic infection with Pseudomonas aeruginosa.

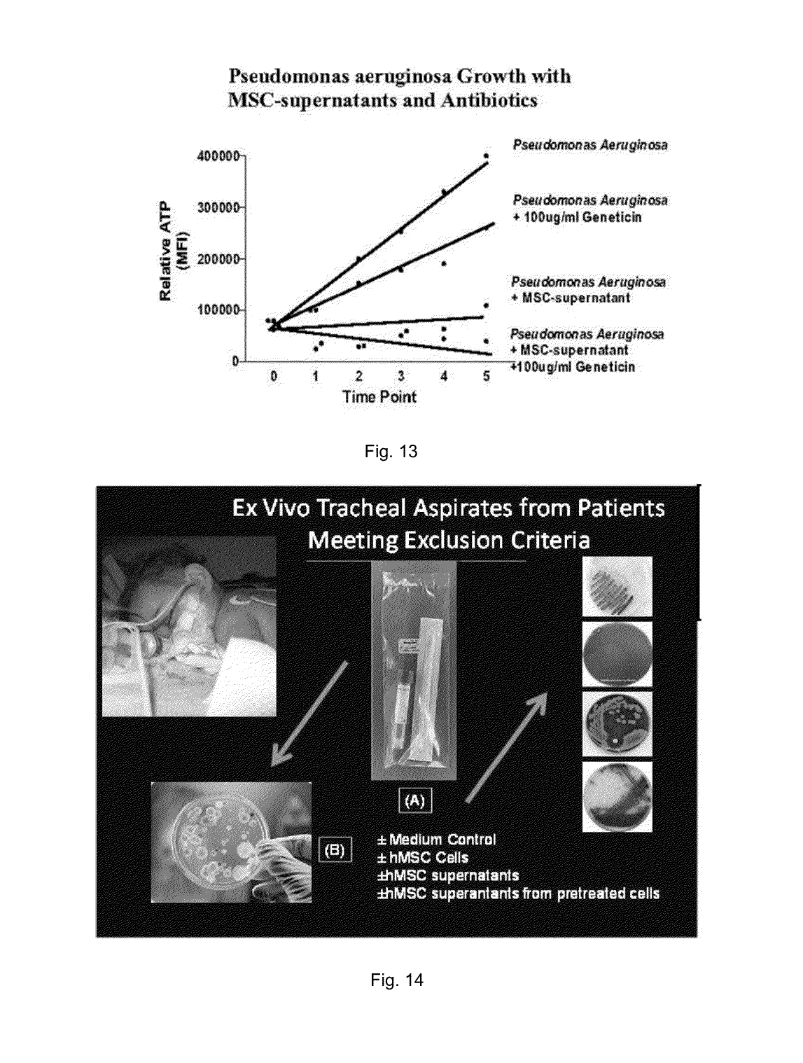

In addition, as shown in FIGS. 12 and 13 of the present application, the inventors have surprisingly discovered that products secreted from MSCs enhance the effectiveness of antibiotics commonly used to treat microbial infections in a subject. It is contemplated that the enhanced antimicrobial efficacy and potency of antimicrobial agents in the presence of MSCs and/or their secreted products allows for the effective treatment of even highly resistant microbial infections using a normally subtherapeutic dose of a given antimicrobial agent. Thus, a wide variety of microbial infections and/or related diseases and disorders may be treated by delivering MSCs and/or their secreted products in combination with one or more antimicrobial agents to a subject in need thereof.

Therefore, an aspect of the application relates to a method for treating a microbial infection in a subject by administering to the subject a therapeutically effective amount of MSCs alone or in a combination therapy with one or more additional antimicrobial agents, wherein the MSCs potentiate the therapeutic activity of the antimicrobial agent.

MSCs for use in the methods and/or pharmaceutical compositions of the application include the formative pluripotent blast or embryonic cells that differentiate into the specific types of connective tissues, (i.e., the tissue of the body that support specialized elements, particularly including adipose, osseous, cartilaginous, elastic, muscular, and fibrous connective tissues) depending on various in vivo or in vitro environmental influences.

MSCs for use with the application may be derived from any human or non-human tissue that provides MSCs capable of producing, expressing, and/or secreting anti-inflammatory and/or anti-microbial factors. The MSCs can be autologous or allogeneic to the subject being treated with the methods of the present application.

Examples of tissue sources include prenatal sources, postnatal sources, and combinations thereof. Tissues for deriving a suitable source of MSCs include, but are not limited to, bone marrow (BM), blood (peripheral blood), dermis, periosteum, synovium, peripheral blood, skin, hair root, muscle, uterine endometrium, adipose or fat, placenta, menstrual discharge, chorionic villus, amniotic fluid and umbilical cord blood and tissue. MSCs may be derived from these sources individually, or the sources may be combined to produce a mixed population of MSCs from different tissue sources.

MSCs for use with the application may comprise purified or non-purified MSCs. MSCs for use with the application include those disclosed in the following references, the disclosures of which are incorporated herein by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal et al., J. Cell Biochem. (1997) 64(2): 295 312; Cassiede et al., J. Bone Miner. Res. (1996) 11(9): 1264 1273; Johnstone et al., (1998) 238(1): 265 272; Yoo, et al., J. Bone Joint Sure. Am. (1998) 80(12): 1745 1757; Gronthos, Blood (1994) 84(12): 416-44173; Basch et al., J. Immunol. Methods (1983) 56:269; Wysocki and Sato, Proc. Natl. Acad. Sci. (USA) (1978) 75: 2844; and Makino et al., J. Clin. Invest. (1999) 103(5): 697 705.

MSCs can be expanded ex vivo prior to use in an embodiment of the present application. For example, MSCs can be derived from the bone marrow of a subject and then maintained in culture. Although the invention is not limited thereof, MSCs can be isolated, preferably from bone marrow or adipose tissue, purified, and expanded in culture, i.e. in vitro, to obtain sufficient numbers of cells for use in the methods described herein. MSCs found in the bone, are normally present at very low frequencies in bone marrow (1:100,000) and other mesenchymal tissues (see Caplan and Haynesworth, U.S. Pat. No. 5,486,359). For example, human bone marrow preparations may be derived from the iliac crest of a subject. Nucleated cells can be isolated from the bone marrow preparations and plated in a suitable growth media. The cells are then passed and maintained in culture media.

“Cultured” and “maintained in culture” are interchangeably used when referring to the in vitro cultivation of cells and include the meaning of expansion or maintenance of a cell population under conditions known to be optimal for cell growth. The cell culture is maintained under culture conditions including suitable temperature, pH, nutrients, and proper growth factors, which favor the in vitro expansion and survival of the immunosuppressive cells. In certain embodiments, MSCs may be harvested and stored (e.g., by cryogen freezing), allowing banking of cells for later use. The terms “Passage” or “Passed” as used herein refer to the process of maintaining a group of cells through a series of cultures. In a specific example, the cells are passed when they are 90%-100% confluent.

In some embodiments of the application, MSCs are grown under conditions that incorporate the use of a culture media that comprises serum. The application may be practiced with serum from any mammal including, but not limited to, human, bovine, goat, pig, horse, rabbit, rat, and combinations thereof. The amount of serum used may vary according to the intended use of the stem cells being cultured. In some embodiments of the application, the immunosuppressive cells are grown in media comprising less than about 5% serum. Some embodiments of the application culture MSCs in medium containing between about 0.1% and 0.2% serum.

As discussed above, MSCs have been found to be environmentally responsive and have the capacity to secrete factors that are both anti-inflammatory and anti-microbial in response to the surrounding milieu in which they reside. Therefore, in some aspects, the antimicrobial enhancing capabilities of MSCs described herein may be further enhanced prior to administration of the cells to a subject by preconditioning the MSCs to stimulate anti-microbial and/or anti-inflammatory protein secretion. Preconditioning MSCs can be achieved by culturing the MSCs in vitro in a specific microenvironment or disease setting corresponding to the intended therapeutic use or infection site.

MSCs can also be preconditioned by culturing the cells in the presence of cytokines commonly found at the site of a microbial infection due to the subject's inflammatory response including, but not limited to, TNFα, IFNγ, IL-1β, IL-8, IL-10, IL-17, and MIP-1α. In some embodiments, MSCs can be preconditioned using inflammatory cytokines found in a cystic fibrosis disease setting. For example, MSCs can be preconditioned by culturing the cells in the presence of inflammatory cytokines, such as IL-8, IL-6, GM-CSF, and ICAM-1, found at elevated levels on the cell surface of, or media from, cystic fibrosis human airway epithelial cells.

In other aspects, MSCs can be preconditioned by culturing the cells in the presence of microbes associated with a microbial infection in the subject to be treated. For example, MSCs are preconditioned by culturing the MSCs in the presence of P. aeruginosa for therapeutic use in a subject having P. aeruginosa pneumonia. MSCs can also be preconditioned by culturing the MSCs in the presence of microbial extracts and/or growth medium used to culture a targeted microbe causing a subject's infection.

In another aspect, MSCs can be preconditioned by culturing the MSCs in the presence of mammalian cells obtained directly from the subject (e.g., from the site proximate to a microbial infection or related inflammation) or cells, medium or cellular extracts used in a model of a particular microbial infection or related disease/disorder. In some aspects, MSCs for use in a method of the application can be cultured in the presence of diseased, infected or damaged cells obtained from a subject having a microbial infection. For example, MSCs can be preconditioned by culturing the cells in the presence of scarred bronchial epithelial cells obtained from a subject having pneumonia associated with cystic fibrosis.

In certain aspects, the application provides methods of administering to the subject a combination therapy including a therapeutically effective amount of MSCs and one or more antimicrobial agents, wherein the MSCs potentiate the therapeutic activity of the antimicrobial agent.

The phrase “combinatorial therapy” or “combination therapy” embraces the administration of MSCs and one or more antimicrobial therapeutic agents as part of a specific treatment regimen intended to provide beneficial effect from the co-action of these therapeutic agents (i.e., the enhanced therapeutic effectiveness of an antimicrobial agent). Administration of these therapeutic agents in combination typically is carried out over a defined period (usually minutes, hours, days or weeks depending upon the combination selected). In some aspects, where the MSCs and the additional antimicrobial therapeutic are administered separately (either in separate compositions administered simultaneously or in separate compositions administered at different time intervals), one would generally ensure that a significant period of time did not expire between the times of each delivery, such that the additional antimicrobial therapeutic and the MSCs would still be able to exert an advantageously combined effect. “Combinatorial therapy” or “combination therapy” is intended to embrace administration of MSCs and one or more therapeutic antimicrobial agents in a sequential manner, that is, wherein the MSCs and the antimicrobial agent are administered at a different time, as well as administration of both the MSCs and one or more antimicrobial agents in a substantially simultaneous manner. Substantially simultaneous administration can be accomplished, for example by administering to the subject an individual dose having a fixed ratio of each of the MSCs and an antimicrobial agent or in multiple, individual doses for each. Sequential or substantially simultaneous administration of MSCs and an antimicrobial agent can be effected by any appropriate route. The therapeutic agents can be administered by the same route or by different routes. The sequence in which the MSCs and the antimicrobial agents are administered is not narrowly critical.

Microbial infections in accordance with this application that can be treated with MSCs alone or in combination with an antimicrobial agent can include any bacterial, fungal or viral infection in a subject. In some aspects, MSCs alone or in combination with an antimicrobial agent are administered to subjects having or at risk of having a microbial infection.

In certain aspects, the microbial infections that can be treated with MSCs alone or in combination with an antimicrobial agent include respiratory microbial infections, especially those commonly associated with cystic fibrosis.

In some aspects, microbial infections in a subject that can be treated with MSCs alone or in combination with an antimicrobial agent (e.g., an antibacterial agent) are bacterial infections. Bacterial infections treated with MSCs alone or in combination with an antibacterial agent can include bacterial infections caused by gram-positive bacteria, such as Staphylococcus and Streptococcus or gram-negative bacteria, such as E. Coli.

In some embodiments, the bacterial infections treated with MSCs alone or in combination with an antibacterial agent can include bacterial infections caused by an antibiotic resistant bacterium. In an exemplary embodiment, a subject that can benefit from treatment with MSCs alone or in combination with an antibiotic agent described herein can be a hospital patient at risk of developing nosocomial infection or a subject known to be infected with or having been exposed to antibiotic resistant bacteria such as, for example, Methicillin-resistant S. aureus (MRSA), Vancomycin-intermediary-sensible S. aureus, and Vancomycin-resistant S. aureus. Methods of detecting the presence of a Staphylococcus bacterial infection are well known, for example, by culturing from a sample from the subject, e.g. a blood culture, can be used.

In another aspect, MSCs alone or in combination with antimicrobial agents described herein can be administered to a subject to inhibit the development of a disease condition or disorder associated with a microbial infection. Exemplary diseases and disorders associated with a bacterial microbial infection can include, without limitation, postoperative wound infections, bacteraemia, septic arthritis, pneumonia, osteomyelitis, meningitis, mastitis, erysipelas, cellulitis, sepsis, acute endocarditis, furuncles, carbuncles, superficial abscesses, deep abscesses in various organs, impetigo, food poisoning, gastroenteritis, urinary tract infection, toxic shock syndrome, and scalded skin syndrome.

In some aspects, the bacterial infection treated in accordance with the present method is a respiratory bacterial infection. In some embodiments, the respiratory bacterial infection is associated with cystic fibrosis. In certain embodiments, the respiratory bacterial infection is pneumonia. In some embodiments, the pneumonia treated is pneumonia associated with cystic fibrosis, such as but not limited to a Pseudomonas aeruginosa, Staphylococcus aureus, or Streptococcus pneumoniae pneumonia associated with cystic fibrosis. In an exemplary embodiment, MSCs can be co-administered with the antibacterial agent geneticin (G418) for the treatment of Pseudomonas aeruginosa pneumonia associated with cystic fibrosis.

Additional microbial infections in a subject that can be treated with MSCs alone or in combination with an antimicrobial agent (i.e., an antifungal agent) include fungal infections. Non-limiting examples of fungal infections treated through a method of the present invention include corneal, lung, skin/nail, mucosal, and systemic fungal infections.

In some embodiments, a method of the present invention can be used to treat corneal fungal infections and related inflammation. For example, in certain embodiments of the present invention, the combination therapy methods may be used to treat fungal keratitis. Fungal keratitis treated in accordance with the present invention may be related to fungal genera including, for example, Fusarium, Penicillium, Aspergillus, Cephalosporium (Acremonium), Curvularia, Alternaria, Trichophyton, Microsporum, Epidermophyton, Scopulariopsis, and Candida.

The methods of the present invention may also be used to treat: lung fungal infections related to fungal genera including for example, Aspergillus and Histoplasma; skin/nail fungal infections (e.g., Athlete's Foot) related to fungal general including for example, Microsporum, Epidermophyton and Trichophyton; mucosal fungal infections related to fungal genera including for example, Candida.; and systemic fungal infections related to fungal genera including for example, Candida and Aspergillus.

Methods of the present invention may also be used in the treatment and prevention of a nosocomial fungal infection (i.e., hospital-acquired fungal infections). In some embodiments, MSCs alone or in combination with an antifungal agent can be administered to a subject who has undergone a medical intervention (e.g., a surgical intervention). In an alternative embodiment, MSCs alone or in combination with an antifungal agent can be administered to a subject prior to the subject undergoing a medical intervention. Additionally, MSCs alone or in combination with an antifungal agent can be administered to a subject both prior to and after the subject has undergone a medical intervention.

In addition, subjects who do not have, but are at risk of developing a fungal infection can be treated according to the methods of the present invention. In such subjects, the treatment can inhibit or prevent the development of fungal infection in the subject. For example, MSCs alone or in combination with and antifungal agent described herein can be administered to neutropenic subjects. Neutropenic subjects can have neutropenia related to current or prior immunosuppressive therapy, an infection (e.g., AIDS) or an otherwise dysfunctional immune system. Neutropenic subjects are predisposed to the development of invasive fungal infections, most commonly including Candida species and Aspergillus species, and, on occasion, Fusarium, Trichosporon and Dreschlera. Cryptoccocus infection is also common in patients on immunosuppressive agents.

In some aspects, microbial infections in a subject that can be treated with MSCs alone or in combination with an antimicrobial agent (e.g., an antiviral agent) are viral infections. Non-limiting examples of viral infections treated through a method of the present invention include corneal, lung, skin/nail, mucosal, and systemic viral infections. In some embodiments, a method of the present invention can be used to treat viral infections associated with ventilator-associated pneumonia (VAP). For example, MSCs alone or in combination with an antiviral agent may be used to treat influenza, parainfluenza (PIV), respiratory syncytial virus (RSV), or Middle East respiratory syndrome coronavirus (MERS-CoV) viral pneumonia.

In certain aspects, the combination therapy of MSCs and one or more antimicrobial therapeutics in a method and/or composition of the present application may reduce the amount of either MSCs or the antimicrobial compound needed as a therapeutically effective dosage, and thereby reduce any negative side effects the agents may induce in vivo. Therefore, the amount of an antimicrobial agent utilized in a method and/or composition of the present application can include a subtherapeutic amount of the antimicrobial agent. That is, an amount that would not be therapeutically effective when not included in a combination therapy with MSCs. In addition, the combination of MSCs with one or more antimicrobial agents in a method and/or composition described herein may reduce the MIC (minimum inhibitory concentration) of the antimicrobial therapeutic, which in turn reduces the opportunity for microbial resistance to specific antimicrobial therapeutics.

The pharmaceutical compositions described herein can be administered by any means that achieve their intended purpose. In some embodiments, a pharmaceutical composition including MSCs alone or in combination with an antimicrobial agent can be administered to the subject systemically. It is contemplated that once administered to a subject, MSCs will home to one or more sites of microbial infection in the subject. In certain embodiments, the MSCs can be delivered to the subject by intravenous injection into blood. In an exemplary embodiment, MSCs can be delivered to the subject via retro-orbital injection of the venous sinus (also referred to as peri-orbital, posterior-orbital and orbital venous plexus administration) where the MSCs rapidly diffuse to the lung tissue of a subject.

Therapeutic compositions of the present application are not limited to systemic administration. Therefore, in other embodiments, therapeutic compositions described herein can be delivered to the subject by injection into or to an area proximate a site of infection and/or inflammation. In still other embodiments, the therapeutic compositions can be delivered to the subject by surgical implantation. In still other embodiments, the therapeutic composition can be delivered to the subject by subcutaneous injection, intra-peritoneal injection, or intra-synovial injection.

In certain embodiments, the MSCs and/or an antimicrobial agent may be inserted into a delivery device which facilitates introduction by injection or implantation into the subjects. Such delivery devices may include tubes or intraluminal devices, e.g., catheters, for injecting cells and fluids into the body of a recipient subject. In a preferred embodiment, the tubes additionally have a needle, e.g., a syringe, through which the cells of the application can be introduced into the subject at a desired location.

Therapeutic MSC and antimicrobial compositions of the application may be prepared for delivery in a variety of different forms. For example, the cells may be suspended in a solution or gel or embedded in a support matrix when contained in such a delivery device. MSCs may be mixed with a pharmaceutically acceptable carrier or diluent in which the MSCs remain viable. Pharmaceutically acceptable carriers and diluents include saline, aqueous buffer solutions, solvents and/or dispersion media. The use of such carriers and diluents is well known in the art. The solution is preferably sterile. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the application may be prepared by incorporating cells as described herein in a pharmaceutically acceptable carrier or diluent and, as required, other ingredients enumerated above, followed by filtered sterilization.

In some aspects, only a single treatment with a combination of MSCs and an antimicrobial agent may be required. Alternatively, multiple administrations of MSCs and/or an antimicrobial agent may be employed. In some aspects, a combination of MSCs and an antimicrobial agent described herein can be administered continuously until infection resolution. MSCs and/or pharmaceutical compositions described herein can be administered prior to a microbial infection, after infection but prior to the manifestation of symptoms of a disease of disorder associated with the infection to prevent further microbial multiplication thereby hindering development of the disease or its progression.

A therapeutically effective amount of MSCs alone or in a combination therapy administered to a subject with an antimicrobial agent can be determined by a practitioner based upon such factors as the age of the subject and/or donor, the mode of administration, the number of, or frequency of administrations, the particular microbial infection to be treated and other variables known to those of skill in the art. In an exemplary embodiment, a therapeutically effective amount of MSCs for the treatment of a microbial infection is the amount or concentration of cells resulting in a significantly increased subject survival rate, attenuated weight loss, decreased clinical scoring, decreased tissue pathology associated with bacterial infection, increased macrophage recruitment to the site of infection and/or decreased leukocyte recruitment to the site of infection. In another embodiment, a therapeutically effective amount of MSCs for the treatment of a microbial infection is the amount or concentration of cells required to potentiate or enhance the therapeutic activity of an antimicrobial agent co-administered to a subject. For example, it is well within the skill of the art to start doses of the MSCs at levels lower than required to achieve the desired therapeutic effect and to gradually increase the dosage until the desired effect is achieved. In some embodiments, the number of MSCs administered is from about 10⁴ to about 10⁸ cells.

Exemplary antibacterial therapeutic agents for use in the methods and/or therapeutic compositions of the application include, but are not limited to, colloidal silver, penicillin, penicillin G, erythromycin, polymyxin B, viomycin, chloromycetin, streptomycins, cefazolin, ampicillin, methicillin, oxacillin, nafcillin, cloxacillin, dicloxacillin azactam, tobramycin, cephalosporins (including cephalothin, cefazolin, cephalexin, cephradine, cefamandole, cefoxitin, and 3rd-generation cephalosporins), carbapenems (including imipenem, meropenem, Biapenem), bacitracin, tetracycline, doxycycline, geneticin, gentamycin, quinolines, neomycin, clindamycin, kanamycin, metronidazole, treptogramins (including Quinupristin/dalfopristun (Synercid™)), Streptomycin, Ceftriaxone, Cefotaxime, Rifampin, glycopeptides (including vancomycin, teicoplanin, LY-333328 (Ortivancin), dalbavancin), macrolides (including erythromycin, clarithromycin, azithromycin, lincomycin, and clindamycin), ketolides (including Telithromycin, ABT-773), tetracyclines, glycylcyclines (including Terbutyl-minocycline (GAR-936)), aminoglycosides, chloramphenicol, Imipenem-cilastatin, fluoroquinolones (including ofloxacin, sparfioxacin, gemifloxacin, cinafloxacun (DU-6859a)) and other topoisomerase inhibitors, Trimethoprim-sulfamethoxazole (TMP-SMX), Ciprofloxacin, topical mupirocin, Oxazolidinones (including AZD-2563, Linezolid (ZyvoX™)), Lipopeptides (including Daptomycin, Ramoplanin), ARBELIC (TD-6424) (Theravance), TD6424 (Theravance), isoniazid (INN), rifampin (RIF), pyrazinamide (PZA), Ethambutol (EMB), Capreomycin, cycloserine, ethionamide (ETH), kanamycun, and p-aminosalicylic acid (PAS).

In some aspects, an antibacterial agent for use in the methods and/or therapeutic compositions of the application can include a beta (β)-lactam antibiotic agent. In general, any beta-lactam antibiotic (a beta-lactam antibiotic is a compound with antibiotic properties and contains a beta-lactun nucleus in its molecular structure) could be used in compositions and methods according to this application. If desired, a suitable derivative of a beta-lactam antibiotic may also be used. Non-limiting examples of suitable derivatives include pro-drugs, metabolites, esters, ethers, hydrates, polymorphs, solvates, complexes, enantiomers, adducts and the like of such beta-lactam antibiotics. Non-limiting examples of typical beta-lactam antibiotics include those belonging to penicillins, penems, carbapenems, cephalosporins, and monobactams. Typical examples beta-lactam antibiotics include, but are not limited to amoxicillin, ampicillin, pivampicillin, hetacillin, bacampicillin, metampicillin, talampicillin, epicillin, carbenicillin, ticarcillin, temocillin, azlocillin, piperacillin, mezlocillin, mecillinam, sulbenicillin, clometocillin, benzathine, benzylpenicillin, procaine benzylpenicillin, azidocillin, penamecillin, propicillin, benzathine phenoxymethylpenicillin, pheneticillin, cloxacillin, dicloxacillin, flucloxacillin, oxacillin, methicillin, nafcillin, faropenem, biapenem, ertapenem, doripenem, imipenem, meropenem, panipenem, cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefinetazole, carbacephem, loracarbef, cefixime, ceftriaxone, ceftazidime, cefoperazone, cefcapene, cefdaloxime, cefdinir, cefditoren, cefetamet, cefinenoxime, cefodizime, cefotaxime, cefpimizole, cefpiramide, cefpodoxime, cefsulodin, cefteram, ceftibuten, ceftiolene, ceftizoxime, oxacephem, flomoxef, latamoxef, cefepime, cefozopran, cefpirome, cefquinome, ceftobiprole, ceftaroline fosamil, ceftiofur, cefquinome, cefovecin, aztreonam, tigemonam, carumonam, tabtoxin, ceftolozane and the like.

Exemplary antifungal agents for use in the methods and/or therapeutic compositions of the application include any of the well known antifungal agents typically falling into one of three main groups. The major group includes polyene derivatives, including amphotericin B and the structurally related compounds nystatin and pimaricin, which are only administered intravenously. These are broad-spectrum antifungals that bind to ergosterol, a component of fungal cell membranes, and thereby disrupt the membranes, leading to cell death. Amphotericin B is usually effective for systemic mycoses, but its administration is limited by toxic effects that include fever and kidney damage, and other accompanying side effects such as anemia, low blood pressure, headache, nausea, vomiting and phlebitis. The unrelated antifungal agent flucytosine (5-fluorocytosine), an orally absorbed drug, is frequently used as an adjunct to amphotericin B treatment for some forms of candidiasis and cryptococcal meningitis. Its adverse effects include bone marrow depression with leukopenia and thrombocytopenia.

The second major group of antifungal agents includes azole derivatives which impair synthesis of ergosterol via lanosterol demethylase and lead to accumulation of metabolites that disrupt the function of fungal membrane-bound enzyme systems (e.g., cytochrome P450) and inhibit fungal growth. Significant inhibition of mammalian P450 results in important drug interactions. This group of agents includes ketoconazole, clotrimazole, miconazole, econazole, butoconazole, oxiconazole, sulconazole, terconazole, fluconazole, Voriconazole, ZD-08070, UK-109496, SCH 56592 and itraconazole. These agents may be administered to treat systemic mycoses. Ketoconazole, an orally administered imidazole, is used to treat nonmeningeal blastomycosis, histoplasmosis, coccidioidomycosis and paracoccidioidomycosis in non-immunocompromised patients, and is also useful for oral and esophageal candidiasis. Adverse effects include rare drug-induced hepatitis; ketoconazole is also contraindicated in pregnancy. Itraconazole appears to have fewer side effects than ketoconazole and is used for most of the same indications. Fluconazole also has fewer side effects than ketoconazole and is used for oral and esophageal candidiasis and cryptococcal meningitis. Miconazole is a parenteral imidazole with efficacy in coccidioidomycosis and several other mycoses, but has side effects including hyperlipidemia and hyponatremia.

The third major group of antifungal agents includes allylamnines-thiocarbamates, which are generally used to treat skin infections. This group includes tolnaftate and naftifine. Another antifungal agent is griseoflulvin, a fungistatic agent which is administered orally for fungal infections of skin, hair or nails that do not respond to topical treatment.

Exemplary antiviral agents for use in the methods and/or therapeutic compositions of the application include, but are not limited to, any antiviral agent commonly used to treat or prevent viral pneumonia, such as but not limited to, Amantadine, Rimantadine, Zanamivir, Oseltamivir Cidofovir lopinavir/ritonavir, ribavirin, RSV immunoglobulin, Palivizumab Acyclovir, Ganciclovir, Foscarnet, Varicella-zoster and intravenous immunoglobulin.

The antimicrobial agents described herein can be provided in a pharmaceutical composition with the MSCs or in a separate composition to be co-administered with the MSCs in accordance with a combination therapy of the application. The pharmaceutical composition can further include a conventional pharmaceutical carrier or excipients, an be provided in solid, semi-solid, liquid or aerosol dosage forms, such as, for example, tablets, capsules, powders, liquids, gels, suspensions, suppositories, aerosols or the like. In addition, these compositions may include additional active therapeutic agents, adjuvants, etc.

For example, pharmaceutical compositions including antimicrobial agents can contain pharmaceutically acceptable carriers, such as excipients and auxiliaries that facilitate processing of the antimicrobial agents into compositions that can be used pharmaceutically. The pharmaceutical compositions can be manufactured in a known manner, such as by conventional mixing, granulating, dragee-making, dissolving, lyophilizing processes, and the like. For example, pharmaceutical compositions for oral use can be obtained by combining the antimicrobial agents described herein with solid excipients, optionally grinding the resulting mixture, and processing the mixture of granules after adding auxiliaries (if desired or necessary) to obtain tablets or dragee cores.

Excipients that can be used as part of the pharmaceutical composition can include fillers, such as saccharides (e.g., lactose or sucrose), mannitol or sorbitol, cellulose preparations and/or calcium phosphates, for example, tricalcium phosphate or calcium hydrogen phosphate, as well as binders, such as starch paste using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as sodium alginate. Auxiliaries can include flow-regulating agents and lubricants, such as silica, talc, stearic acid or salts thereof, such as magnesium stearate or calcium stearate, and/or polyethylene glycol. Dragee cores can be provided with coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. To produce coatings resistant to gastric juices, solutions of suitable cellulose preparations, such as acetylcellulose phthalate or hydroxypropylmethylcellulose phthalate can be used. Slow-release and prolonged-release formulations may be used with particular excipients, such as methacrylic acid-ethylacrylate copolymers, methacrylic acid-ethyl acrylate copolymers, methacrylic acid-methyl methacrylate copolymers, and methacrylic acid-methyl methylacrylate copolymers. Dye stuffs or pigments can be added to the tablets or dragee coatings, for example, for identification or to characterize combinations of active compound doses.

Other pharmaceutical preparations that can be used orally include push-fit capsules made of gelatin, as well as soft, sealed capsules made of gelatin and a plasticizer, such as glycerol or sorbitol. The push-fit capsules can contain the active compounds in the form of granules that may be mixed with fillers, such as lactose, binders, such as starches, and/or lubricants, such as talc or magnesium stearate and, optionally, stabilizers. In soft capsules, the active compounds can be dissolved or suspended in suitable liquids, such as fatty oils or liquid paraffin. In addition, stabilizers may be added.

Examples of formulations for parenteral administration can include aqueous solutions of antimicrobial agents in water-soluble form, for example, water-soluble salts and alkaline solutions. Especially preferred salts are maleate, fumarate, succinate, S,S tartrate, or R,R tartrate. In addition, suspensions of the active compounds as appropriate oily injection suspensions can be administered. Suitable lipophilic solvents or vehicles can include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, such as sodium carboxymethyl cellulose, sorbitol, and/or dextran. Optionally, the suspension may also contain stabilizers.

In further aspects of the invention, an MSC and an antimicrobial agent combination therapy of the invention can be administered to a subject in conjunction with additional pharmaceutically active agents, such as an anti-inflammatory agent. Examples of additional anti-inflammatory agents include, but are not limited to, non-steroidal anti-inflammatory drugs (NSAIDs) acetaminophen, salicylate, acetyl-salicylic acid (aspirin, diflunisal), ibuprofen, Motrin, Naprosyn, Nalfon, and Trilisate, indomethacin, glucametacine, acemetacin, sulindac, naproxen, piroxicam, diclofenac, benoxaprofen, ketoprofen, oxaprozin, etodolac, ketorolac tromethamine, ketorolac, nabumetone, and the like, and mixtures of two or more of the foregoing. Other suitable anti-inflammatory agents include methotrexate. Examples of steroidal anti-inflammatory agents include, but are not limited to, hydrocortisone, prednisone, prednisolone, methylprednisolone, dexamethasone, betamethasone, and triamcinolone.

Additional pharmaceutically active agents administered to a subject in conjunction with the MSCs and antimicrobial agent combination therapy described herein can include beta adrenergics which include bronchodilators including albuterol, isoproterenol sulfate, metaproterenol sulfate, terbutaline sulfate, pirbuterol acetate and salmeterol formotorol; steroids including beclomethasone dipropionate, flunisolide, fluticasone, budesonide and triamcinolone acetonide.

Anti-inflammatory drugs used in conjunction with a combination therapy of the application for treatment of respiratory microbial infections can include steroids such as beclomethasone dipropionate, triamcinolone acetonide, flunisolide and fluticasone. Other examples of anti-inflammatory drugs include cromoglycates such as cromolyn sodium. Other respiratory drugs, which would qualify as bronchodilators, include anticholenergics including ipratropium bromide.

Additional pharmaceutically active agents administered to a subject in conjunction with the MSCs and antimicrobial agent combination therapy described herein can include antihistamines. Exemplary antihistamines for use in conjunction with MSC and antimicrobial agent combination therapies of the application include, but are not limited to, diphenhydramine, carbinoxamine, clemastine, dimenhydrinate, pryilamine, tripelennamine, chlorpheniramine, brompheniramine, hydroxyzine, cyclizine, meclizine, chlorcyclizine, promethazine, doxylamine, loratadine, and terfenadine. Particular anti-histamines include rhinolast (Astelin), claratyne (Claritin), claratyne D (Claritin D), telfast (Allegra), zyrtec, and beconase.

Percutaneous devices (such as catheters) and implanted medical devices (including, but not limited to, pacemakers, vascular grafts, stents, and heart valves) commonly serve as foci for bacterial infection. The tendency of some microorganisms (e.g., Staphylococcus bacteria) to adhere to and colonize the surface of the device, promotes such infections, which increase the morbidity and mortality associated with use of the devices. Therefore, in another aspect, MSCs alone or in combination with an antimicrobial agent can be used to inhibit bacteria growth on or associated with a medical device by contacting the device with MSCs alone or in combination with one or more antimicrobial agents in an amount effective to inhibit microbial growth.

A medical device according can include any instrument, implement, machine, contrivance, implant, or other similar or related article, including a component or part, or accessory which is: recognized in the official U.S. National Formulary the U.S. Pharmacopoeia, or any supplement thereof; intended for use in the diagnosis of disease or other conditions, or in the cure, mitigation, treatment, or prevention of disease, in humans or in other animals; or, intended to affect the structure or any function of the body of humans or other animals, and which does not achieve any of its primary intended purposes through chemical action within or on the body of human or other animal, and which is not dependent upon being metabolized for the achievement of any of its primary intended purposes.

A medical device can include, for example, endovascular medical devices, such as intracoronary medical devices. Examples of intracoronary medical devices can include stents, drug delivery catheters, grafts, and drug delivery balloons utilized in the vasculature of a subject. Where the medical device comprises a stent, the stent may include peripheral stents, peripheral coronary stents, degradable coronary stents, non-degradable coronary stents, self-expanding stents, balloon-expanded stents, and esophageal stents. The medical device may also include arterio-venous grafts, by-pass grafts, penile implants, vascular implants and grafts, intravenous catheters, small diameter grafts, artificial lung catheters, electrophysiology catheters, bone pins, suture anchors, blood pressure and stent graft catheters, breast implants, benign prostatic hyperplasia and prostate cancer implants, bone repair/augmentation devices, breast implants, orthopedic joint implants, dental implants, implanted drug infusion tubes, oncological implants, pain management implants, neurological catheters, central venous access catheters, catheter cuff, vascular access catheters, urological catheters/implants, atherectomy catheters, clot extraction catheters, PTA catheters, PTCA catheters, stylets (vascular and non-vascular), drug infusion catheters, angiographic catheters, hemodialysis catheters, neurovascular balloon catheters, thoracic cavity suction drainage catheters, electrophysiology catheters, stroke therapy catheters, abscess drainage catheters, biliary drainage products, dialysis catheters, central venous access catheters, and parental feeding catheters.

The medical device may additionally include either arterial or venous pacemakers, vascular grafts, sphincter devices, urethral devices, bladder devices, renal devices, gastroenteral and anastomotic devices, vertebral disks, hemostatic barriers, clamps, surgical staples/sutures/screws/plates/wires/clips, glucose sensors, blood oxygenator tubing, blood oxygenator membranes, blood bags, birth control/IUDs and associated pregnancy control devices, cartilage repair devices, orthopedic fracture repairs, tissue scaffolds, CSF shunts, dental fracture repair devices, intravitreal drug delivery devices, nerve regeneration conduits, electrostimulation leads, spinal/orthopedic repair devices, wound dressings, embolic protection filters, abdominal aortic aneurysm grafts and devices, neuroaneurysm treatment coils, hemodialysis devices, uterine bleeding patches, anastomotic closures, aneurysm exclusion devices, neuropatches, vena cava filters, urinary dilators, endoscopic surgical and wound drainings, bandages, surgical tissue extractors, transition sheaths and dialators, coronary and peripheral guidewires, circulatory support systems, tympanostomy vent tubes, cerebro-spinal fluid shunts, defibrillator leads, percutaneous closure devices, drainage tubes, bronchial tubes, vascular coils, vascular protection devices, vascular intervention devices including vascular filters and distal support devices and emboli filter/entrapment aids, AV access grafts, surgical tampons, and cardiac valves.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

### Mice

All experiments used the congenic B6.129S6-Cftr^{tm2Uth} (R117H/R117H mutation) and C57BL/6J controls (WT). Cftr^{tm2Uth} mice are a type IV Cftr mutant which predominantly affects the pulmonary response to infections. These animals were chosen to specifically investigate the potential proof of concept toward treating the pulmonary consequences of CF with cell based therapies. To study the therapeutic potential of hMSCs we used a sub-lethal model of airway infection and inflammation without the confounding contribution of gastrointestinal fragility. When chronically infected with Pseudomonas aeruginosa, the Cftr^{tm2Uth} mice demonstrated cachexia, weight loss and bronchoalveolar lavage (BAL) changes in cellular differential and cytokines without significant mortality. We have used 3 different groups of Cftrtm2Uth or controls mice for each of 3 different hMSC preparations. In each experiment, we utilized 5-8 mice. In a smaller set of studies (n=2, 5-7 animals/group) we had the availability to explore the use of hMSCs in Cftr^{tm1Uth} (DF508, B6.129S6-Cftr^{tm1Kth}) mice at day 3. The Cftr^{tm1Kth} mice (a type II mutation of Cftr) have all of the manifestations of the CF knockout mouse with gastrointestinal blockage and hypersensitivity to bacterial infection. The groups included both Cftr deficient and WT mice: infected without cell based therapy, infected with hMSCs or infected with WT BMM.

### Murine Model of CF Infection and Inflammation

To generate a transient chronic infection, mice were infected with 5×10⁵ colony-forming units (CFU) Pseudomonas aeruginosa, strain PA-M5715 (a mucoidal clinical isolate) embedded in agarose beads and suspended in 20 μL of PBS. All preparations of Pseudomonas aeruginosa impregnated beads were evaluated for relative colony forming units (CFUs) prior to inoculation into the mice. Cftr^{tm2Uth}, Cftr^{tm1Kth} and WT mice were anesthetized and then inoculated with bacteria into the trachea with a plastic catheter angled toward the right mainstream bronchus. Brochoalveolar lavage (BAL) and whole lung homogenates were evaluated for CFUs at either day 3 or day 10 (post-hMSCs).

### Lung Inflammation

Mice were injected with ketamine (80 mg/kg) and xylazine (10 mg/kg) as previously described. The thoracic cavity was opened and the lungs exposed followed by insertion of a cannula through the trachea into the bronchi and infusion of 1×1 ml of warm PBS containing 0.2% lidocaine to do the BAL. The BAL fluid sample was recovered by aspirating the liquid with a syringe for total cell count, cellular differential. BAL fluid was aliquoted and analyzed for cytokines involved in CF pathophysiology by Luminex multi analyte technology and LL37 by a commercial kit (Hycult Biotech, Plymouth Meeting, Pa. Cat# HK321).

### Clinical and Lung Pathology Scores

Mice were assessed daily for clinical score which was based upon coat quality, posture, ability to right themselves after being placed in lateral recumbence, ambulation and body weight. At 3 days, 10 days, post-mortem or at sacrifice lungs were isolated and assessed for gross lung pathology in addition to quantitative bacteriology. Both gross lung pathology and clinical scores were done by two different individuals who did not know the identity of the treatment groups.

### Lung Histopathology

Concurrent studies were evaluated for lung pathology without BAL using hematoxylin and eosin to define inflammation.

Human Mesenchymal Stem Cells (hMSCs) and Bone Marrow Derived Macrophages (BMM)

hMSCs were obtained from bone marrow aspirates of healthy volunteers after written and verbal informed consent. All procedures were approved by Case Western Reserve. hMSCs from the bone marrow of healthy volunteers were isolated, cultured and immunophenotyped as described previously. hMSCs were used during log-growth. BMMs were generated as previously described. Briefly, hematopoietic progenitors were obtained from the femurs of C57BL/6J. Cells were grown in culture for 7-10 days in the presence of L929 spent medium. hMSCs and BMM were administered at 10⁶/100 ul PBS through the retro-orbital sinus consistent with previous published observations of using this route of administration.

### Macrophages for In Vitro Inflammation Studies

C57BL/6J or Cftrtm2Uth BMM were generated as described above. Once a monolayer was generated and the cells were differentiated, the BMM were grown in the presence and absence of 0.5 μg/ml lipopolysaccharide to induce inflammatory cytokines TNFα and IL-6 in vitro. Stimulated BMM were cultured in vitro with or without the addition of supernatants derived from cultured hMSCs. After 24 hours, cells were harvested and evaluated for TNFα or IL-6 gene expression based upon the changes in cytokine production in the mouse model post-hMSC administration. The experiments were done with hMSC supernatants from 3-different donors and all measurements were done in triplicate.

### Human CF Epithelial Cells

CF epithelial cells have been demonstrated to be hyper responsive to bacterial exposure resulting in elevated production of IL-8 and IL-6. Many cell lines are available to study the CF airway epithelial cell inflammatory response. The studies outlined in this manuscript utilized immortalized cell lines developed by transforming human airway tracheal epithelial cells from a CF patient with adenovirus. The immortalized CF cell line is called (IB3-1 cells), the control cells are the same CF derived tracheal epithelial cells transfected with adenovirus with full-length functional CFTR. These control cells are designated S9 (HC) cells. These cells were kindly provided by the laboratory of Dr. Pamela Davis (Case Western Reserve University, Cleveland, Ohio). Cells were maintained in a 5% CO2 incubator at 37° C. using LHC-8 media (Biosource, Camarillo, Calif.). All media contained penicillin/streptomycin and 10% fetal bovine serum. The experiments were done at least 3-times with 3-different donors of 48 hour-cultured hMSC supernatants. After 24 hours, cells were harvested and evaluated for IL-8 or IL-6 based upon the changes in cytokine production in the mouse model post-hMSC administration.

### Bactericidal Assays

Pseudomonas aeruginosa (PA M5715, a clinical isolate) was streaked on Tryptic Soy agarose (TSA) plates then inoculated into flasks. PA M5715 was plated at dilutions of 10⁶ to 10⁹ to define the appropriate dilutions. Growth curve analysis and viability was used to define the CFUs. PA M5715 dilutions (10⁴ to 10⁷) were mixed 1:1 with either PBS, un-stimulated hMSC culture medium (US) or LPS stimulated hMSC culture medium (LPS, 0.5 μg/ml for 24 hours) for 30 minutes at room temperature followed by plating on TSA plates and incubation overnight at 37.5° C. Colony counts were quantitatively assessed at 24 hours.

### Cytokine Gene Expression

Total RNA is extracted by RNAeasy protocol (Qiagen, Valencia, Calif.). Expression of mRNA is determined by RT-PCR using the ABI Prism 7000 Detection System (Applied Biosystems Inc., ABI, Foster City, Calif.). RNA specimens were analyzed in duplicate and normalized to GAPDH. Primers (TNFα, IL-6, IL-8 for either mouse or human samples) were purchased from ABI and validated prior to studies.

### Chemotaxis Assays

BAL derived alveolar macrophages (AM) and peritoneal neutrophils (P) were evaluated for the ability to respond to MSC supernatants (n=3 different donor derived supernatants, using transwells (0.4 μM, Corning, N.Y.). Both alveolar macrophages and neutrophils were obtained as previously described. MSC supernatants were put into the lower chamber and cells were put into the upper chamber as previously described. After 4 hours the numbers of cells in the upper chamber and the lower chamber were counted to reflect the ability to respond to MSC supernatant.

### Statistics

Data were analyzed using quantitative and group comparisons with respect to measurements at individual time points. Data are described using means, standard deviations, and appropriate percentiles including medians and extreme values. Graphical representations show the data within different groups and at different time points. For group and time point comparisons, we used analysis of variance (ANOVA) or F values for variance between the means. For pair-wise comparisons non-parametric Mann-Whitney t-test or Wilcoxon signed-rank test were performed for samples with un-equal numbers or variance respectively, as indicated in the figure legend. Data of significance were established based upon an alpha value of 5% and below (P≦0.05). In some cases we designated alpha values which are not statistically significant as defined by our guidelines, however alpha values approaching 5% are indicative of how close to significance the data is, particularly when dealing with data that does not follow Gaussian distributions and is often found when dealing with in vivo studies. In this case we designate a specific P value with a dot (•P=) instead of a star that is used for significance.

### Results

hMSC Decreases Weight Loss and Lung Pathology Associated with Chronic Pseudomonas aeruginosa Infection

Animals genetically modified to have altered expression of Cftr have been used extensively to study the pulmonary response to chronic infection with Pseudomonas aeruginosa. The benefits of the murine model are that it is a consistent, reproducible model of infection/inflammation in the context of deficient Cftr and normal mucocilliary clearance. Our CF animal CORE Center has done extensive studies on the kinetics of the Cftr deficient lung response to pathogen exposure using a variety of murine Cftr deficient models. The animals used in this study comprised a model that does not have severe gastrointestinal phenotype (Cftr^{tm2Uth}) to explore the potential therapeutic application of hMSCs in resolving the pulmonary manifestations associated with chronic airway infection and inflammation in CF. Cftr^{tm2Uth} mice and control animals were inoculated with Pseudomonas aeruginosa-laden agar beads with and without retro-orbital administration of 10⁶ hMSCs or BMM and followed for either 3 or 10 days. FIG. 1 shows the mean weight loss of Cftr^{tm2Uth} mice in response to Pseudomonas aeruginosa infection with and without treatment with 10⁶ hMSCs or BMM. The mice were followed for 10 days with daily weights and clinical scores. At day 3, the Cftr^{tm2Uth} animals given Pseudomonas aeruginosa lost significant weight when compared to C57Bl/6 mice given the same batch and dosing of Pseudomonas aeruginosa (FIG. 1A, n=3 different experiments). Animals given 10⁶ hMSCs or BMM retro-orbitally initially lost weight but by 3-day began to resolve the cachexia (FIG. 1A, n=3 different hMSC or BMM preparations, P≦0.05). By day 10, the Cftr^{tm2Uth} mice treated with hMSCs had weights comparable to WT mice infected at the same time (FIG. 1B, n=3 different hMSC preparations, P≦0.05). Treatment with BMM caused a statistical increase (P≦0.05) in weight gain over the WT controls and the Cftr^{tm2Uth} at day 10 consistent with the important role of myeloid cells in the CF lung response to infection.

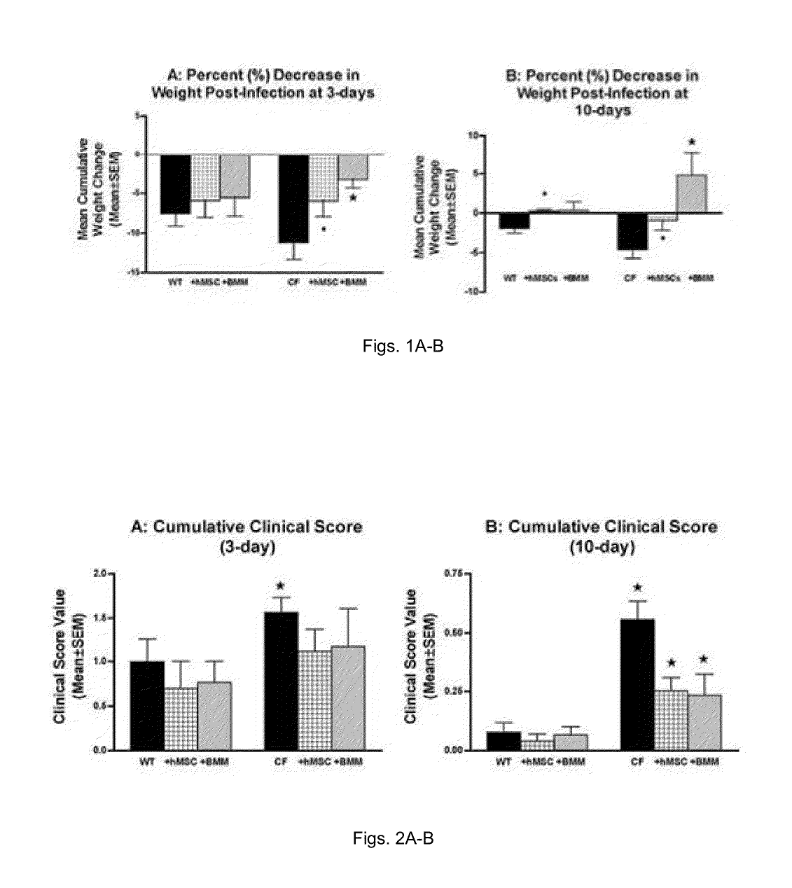

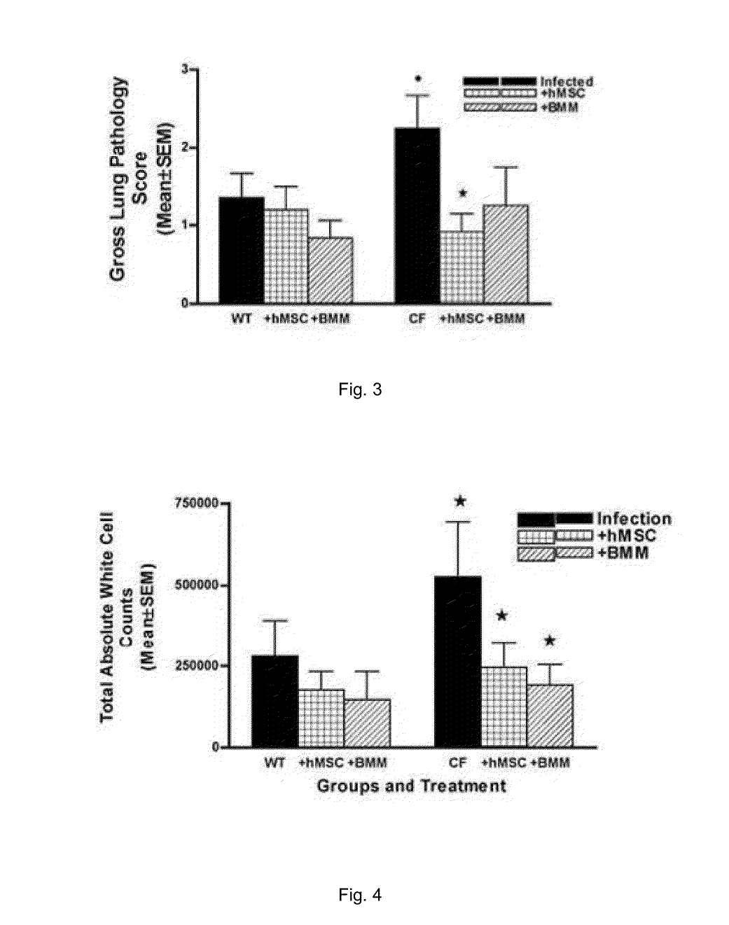

Treatment of Cftr^{tm1Kth} mice (delF508) with hMSCs also statistically decreased the amount of weight loss relative to the untreated infected control showing the consistency of the hMSC effect. Weight loss is often used as a parameter in conjunction with clinical score. When the animals were assessed for clinical score, Cftr^{tm2Uth} mice with infection but without hMSCs had a higher clinical score (P≦0.05) than controls which was attenuated with hMSCs even by 3-days (the higher the value the sicker the animals, FIG. 2A, P=0.07), suggesting a trend toward improvement. At day 10, there was a statistical difference between the WT and Cftr^{tm2Uth} mice in terms of clinical score (FIG. 2B, P≦<0.05) which was significantly decreased by hMSC or BMM therapy (P≦0.05). The improvement in clinical score also reached significance in our Cftrt^{m1Kth} mice. When animals were sacrificed and evaluated for gross lung pathology (FIG. 3), Cftr^{tm2Uth} animals tended to have a greater focal lung consolidation and pathology scores compared to C57Bl/6J controls (FIG. 3, P=0.07,). Administration of the hMSCs statistically changed the pathology scores in the Cftr^{tm2Uth} to have levels comparable with C57BL/6 mice infected with Pseudomonas aeruginosa (P≦0.05). Treatment of the Cftr^{tm2Uth} and WT mice with BMM had no statistical impact on lung pathology. Similar results were also found in the studies using Cftrtm1Kth mice.

hMSC Impact on Lung Inflammation

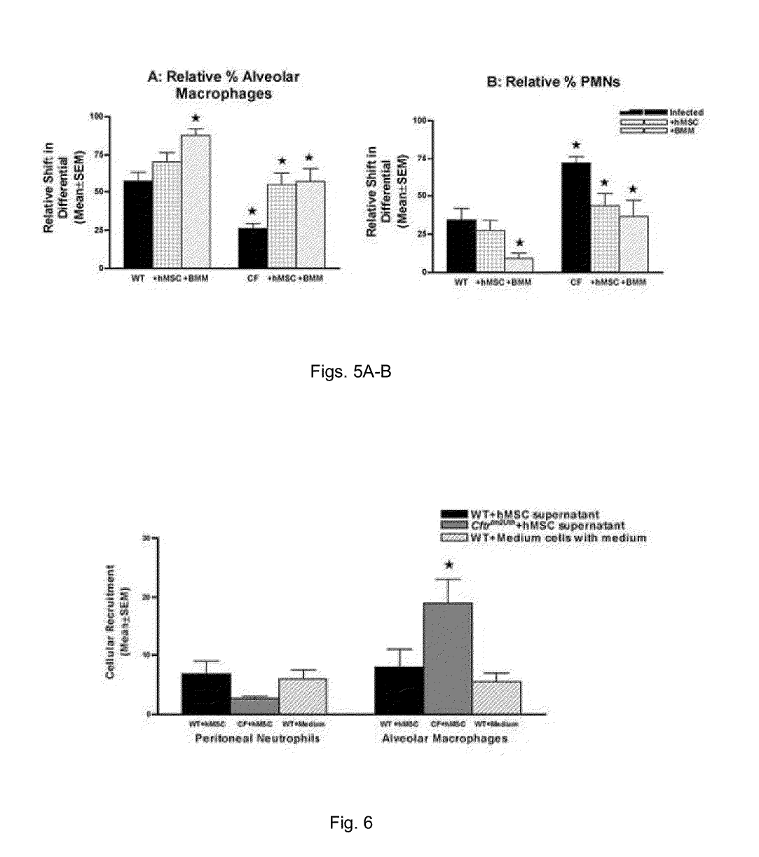

In order to investigate how the hMSCs impact the murine model of CF lung infection and inflammation, animals were euthanized followed by BAL for differentials, and total cell counts. As has been published previously, Cftr^{tm2Uth} animals had higher numbers of BAL leukocytes than WT mice given the same inoculums (FIG. 4, P≦0.05). Cell based therapy, whether it was hMSCs or BMM resulted in a statistical decrease in the overall numbers of BAL leukocytes (P≦0.05, for BMM and hMSCs) in the Cftr^{tm2Uth} which was not observed in the WT controls. The leukocyte recruitment in the Cftr^{tm2Uth} showed decreased relative numbers of alveolar macrophages (FIG. 5A, P≦0.05) and increased numbers of neutrophils (FIG. 5, P≦0.05). Both hMSCs and BMM enhanced recruitment of alveolar macrophages (FIG. 5A, P≦0.05) while attenuating the relative numbers of neutrophils (FIG. 5B, P≦0.05). We obtained similar observations in the Cftr^{tm1Kth} model. To determine if the hMSCs had the capacity to contribute to the shift in inflammatory cell recruitment away from neutrophils toward alveolar macrophages, chemotaxis studies were performed using alveolar macrophages and peritoneal neutrophils from WT and Cftr^{tm2Uth} mice (FIG. 6). There was no difference between WT (FIG. 6) and Cftr^{tm2Uth} (data not shown) neutrophils or alveolar macrophages when cultured with medium alone. Using three different hMSC derived supernatants, there was a significant chemotactic effect of the supernatants on recruiting Cftr^{tm2Uth} alveolar macrophages but not on any other cell type (P≦0.05), with a suggestive suppressive effect on neutrophil recruitment.

hMSC Impact on the Cytokine Response in the In Vivo Murine Model of CF Airway Infection and Inflammation

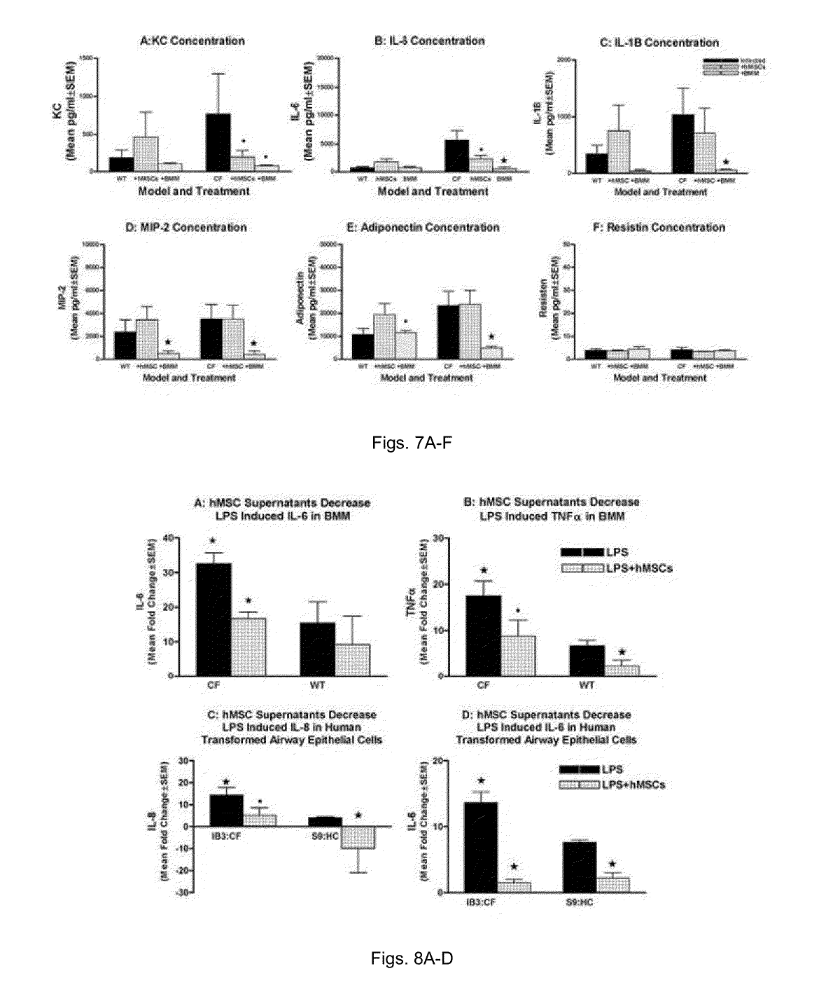

Cytokines are essential in the process of leukocyte recruitment and define the cell type and inflammatory response. We used Luminex multi-analyte technology to measure cytokines known to be involved in CF. KC, IL-6 and IL-1β were measured for chronic inflammation and neutrophil recruitment (FIG. 7). MIP-2, resistin and adiponectin were measured due to their implications in regulating macrophage responses and inflammation. Not surprisingly, Cftr^{tm2Uth} mice had elevated levels of KC (7A), IL-6 (7B), IL-1β (7C). Both KC and IL-6 levels were attenuated by both hMSC and BMM cell based therapy (P≦0.05). Only BMM attenuated the IL-1β levels (7C, P≦0.05). Cftr^{tm2Uth} mice had elevated BAL adiponectin levels (7D) but not MIP-2 (7E) or resistin (7F) compared to infected WT controls. Only BMM attenuated MIP-2 and adiponectin concentrations (P≦0.05). Resistin concentrations are shown for comparison of a non-response. We did not detect IL-10 in any of the BAL samples (data not shown).

Mechanism of hMSC Anti-Inflammatory Activity

To investigate the mechanisms behind the anti-inflammatory impact of the hMSCs and whether it is related to hMSC soluble products, we used two different in vitro assays of cytokine production: LPS stimulation of bone marrow derived macrophages and epithelial cells. In the first set of studies we obtained bone marrow cells from WT and Cftr^{tm2Uth} mice and differentiated them into bone marrow derived macrophages (BMM) in vitro. The WT and Cftrtm2Uth BMM preparations were stimulated with 0.5 μg/ml LPS for 24 hours to induce the production of pro-inflammatory cytokines TNFα and IL-6 mRNA expression. The LPS treated cultures were evaluated with or without the addition of hMSC supernatants to determine if hMSC soluble products could decrease the pro-inflammatory response to LPS in these in vitro cultures of BMM. We measured mouse IL-6, TNF-α mRNA expression and as predicted, LPS stimulated both IL-6 (FIG. 8A, P≦0.05) and TNFα (FIG. 8, P≦0.05) with more being expressed by Cftr^{tm2Uth}

BMM. hMSC supernatants decreased both IL-6 (FIG. 8A, P≦0.05) and TNF-α (FIG. 8B, P=0.07) mRNA expression. Since primary epithelial cells are difficult to culture from CF and WT mice, we took advantage of established human airway epithelial cell models of CF and healthy controls (HC; see description in the methods). LPS stimulation of the CF and HC cells showed elevated IL-8 (FIG. 8C) and IL-6 (FIG. 8D) gene expression, again with the CF cells expressing significantly more of both cytokines (P≦0.05). hMSC supernatant suppressed both IL-8 and IL-6 mRNA expression (P≦0.05). These data suggest that soluble products produced by hMSC contribute to decreased cytokine production in both macrophages and epithelial cells.

hMSCs and Anti-Bacterial Properties

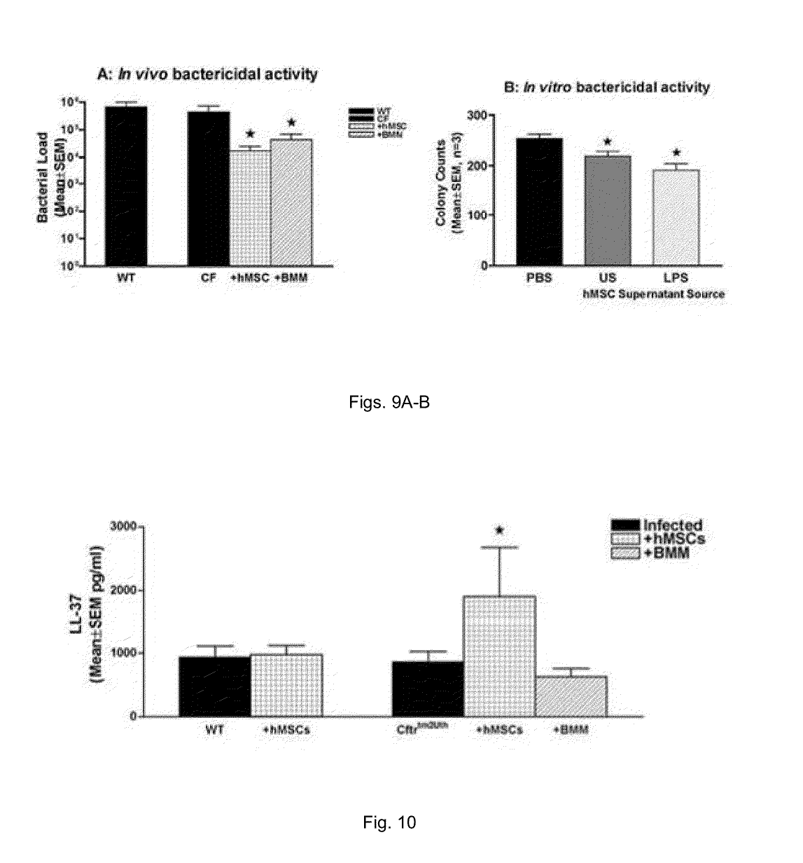

We evaluated the colony forming units of Pseudomonas aeruginosa remaining in the lungs of the Cftrtm2Uth at day 3 to test this function in our in vivo model. Whole lung homogenates were made of the animals and cultured overnight on TSA plates. Both WT and CF animals had significant and comparable bacterial loads at day 3 (FIG. 9A). CF animals treated with hMSCs or BMM, had significantly less bacterial counts (FIG. 9A, P≦0.05), than the animals infected at the same time but without cell based therapy. These observations were consistent in our Cftr^{tm1Kth} animal studies. To determine if this was a direct effect of the hMSCs, supernatants from hMSCs were cultured with Pseudomonas aeruginosa in vitro. The supernatants were derived from hMSCs with or without stimulation with LPS (0.5 μg/ml for 24 hours) to determine if the hMSCs would generate products with enhanced bactericidal activity in response to endotoxin. The Pseudomonas aeruginosa was cultured with the different hMSC supernatants and then plated out on TSA plates and allowed to grow overnight. Bacterial counts were evaluated and compared to controls of bacteria not treated or treated with PBS. The hMSC supernatant obtained from non-stimulated hMSCs (US) and LPS-stimulated hMSC (LPS) culture supernatant significantly decreased the ability of the bacteria to grow in vitro (FIG. 9B, P≦0.05) over the PBS control. BMM supernatants also decreased bacterial load in vitro (data not shown), but is the focus of a separate manuscript. To identify the potential agent associated with the anti-inflammatory and anti-bacterial properties of hMSC supernatants, we evaluated the BAL supernatants from both Cftr^{tm2Uth} and Cftr^{tm1Kth} (data not shown) and controls for the presence of LL-37, because it has been reported to be both anti-inflammatory and antimicrobial. BAL fluid was obtained from mice chronically infected with Pseudomonas aeruginosa with and without hMSC or BMM therapy (FIG. 10, n=3 experiments with 4-6 BAL samples/group). All of the BAL fluid had detectable levels of LL-37, with the highest levels found in the Cftr deficient animals treated with husks (P≦0.05) using the F-test for analysis of variance, P=0.07 for the Mann-Whitney t-test). The trend of increased levels of LL-37 in both Cftr deficient models supports the potential role of LL-37 in the effectiveness of hMSCs at decreasing bacterial load and inflammation. Although the BMM had anti-inflammatory and antimicrobial effects in vivo, they did not appear to associated with LL-37 levels, suggesting the involvement of other anti-microbial proteins besides LL-37 maybe important in the anti-microbial effects of BMM. This is the focus of on-going studies in our laboratory.

Potential Sources of hMSCs In Vivo

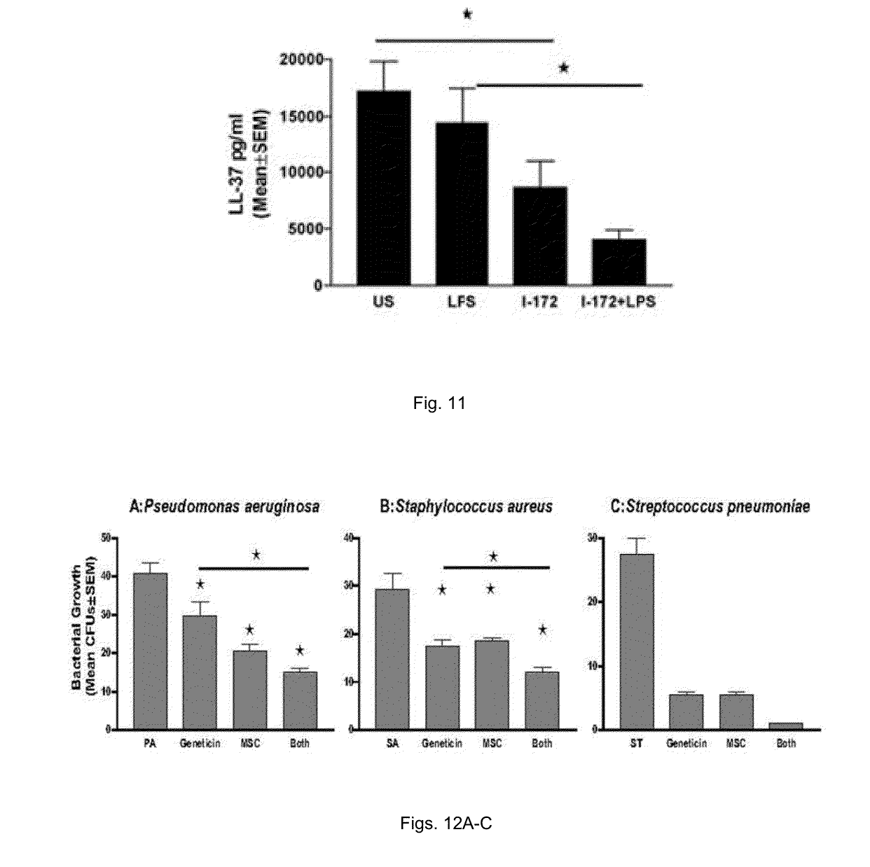

With differences in LL-37 concentrations in the in vivo model, we investigated the potential source of the LL-37. hMSCs cultured in vitro for 24 hours secreted LL-37 (FIG. 11). LPS stimulation for 24 hours did not appear to significantly change the amount of secreted LL-37 relative to the un-stimulated control. Incubation of the hMSCs with the CFTR inhibitor I-172 (10 ug/ml for 48 hours), significantly reduced the ability of hMSCs to secrete LL-37 relative to the un-stimulated control (P≦0.05). Further, when cells were stimulated with LPS after inhibiting CFTR activity (for 48 hours), the amount of LL-37 was even further suppressed relative to the LPS control (P≦0.05). These data suggest that the hMSCs express functional CFTR and that blocking CFTR impacts the ability to produce LL-37 and the ability to respond to LPS. To determine if CF MSCs express CFTR to validate the I-172 studies, we took advantage of an immortal mouse derived MSC clone BMC9, especially since we have a highly reproducible mouse Cftr gene expression assay in our CF animal CORE center. These cells are abundant and have s a MSC phenotype when grown at 37° C. Our data showed that the BMC9 cells have 0.36±0.14% Cftr expression (Ct value of 31±1) compared to intestinal epithelium (Ct value around 20±3), which expresses extremely high levels of Cftr. The sensitivity and specificity of our Cftr expression assay is 40±2 Ct. If we had used a lower expressing tissue, the % of Cftr levels would be higher. BMC9 cells cultured with I-172 (10 μg/ml, for 48 hours) secreted 37±13% (Mean±SEM, n=3) less LL-37 than BMC9 cells not cultured with I-172 (P≦0.05). These data suggest that MSCs express CFTR and that CFTR function impacts the ability of MSCs to produce products such as LL-37. Our future studies are aimed at studying the differences between and Cftr deficient MSCs and control MSCs.

Recently, our laboratory surprisingly established that MSCs are environmentally responsive having the capacity to secrete factors that are both anti-inflammatory and anti-microbial, attenuating inflammation while at the same time aiding in infection resolution associated with Pseudomonas aeruginosa infection (Stem Cell Discovery, In Press. 2013).

### Example 2

### Aim 1. Defining the Antimicrobial and Antibiotic Enhancing Potency, Efficacy and Sustainability of MSC Derived Products Against Bacteria In Vitro

### Rational

Bacterial infections are a major cause of morbidity and mortality in pediatric patients. Compliance in using antibiotics, antibiotic effectiveness and the development of antibiotic resistant strains are all major issues involved with these infections. MSCs and their products have been shown to antimicrobial activity. We have further been able to demonstrate that MSCs enhance antibiotic potency (FIG. 12). This aim focuses on defining the MSCs product potency and the antibiotic enhancing effect against the more common pathogens associated with pneumonia and sepsis.

In the first part of this aim we will specifically focus on the following bacteria: Pseudomonas aeruginosa, Staphylococcus aureus and Streptococcus pneumonia, since these are the most common pathogens associated with severe chronic pneumonia. Each of the bacteria will be cultured with and without MSCs or MSC products (10⁶, 10⁵, 10⁴ MSCs or dilutions of 1:1, 1:2 and 1:5 of the MSC products) to evaluate the change in growth and viability kinetics of each pathogen. The treatment combinations will be done for 2, 4, 24, 48 and 72 hours to determine the duration of the antimicrobial and antibiotic enhancing effect. The conditions will include growth with: no MSC products, pathogen specific antibiotics, MSC secreted products and a combination of MSC secreted products and antibiotics. The bacteria and their specific antibiotics and dosages are outlined in Table I. At each time point, an aliquot of bacteria from each condition will be removed and analyzed for growth properties and viability described in the next paragraph. The antibiotic free MSC products will be derived from MSC cells cultured in vitro for 72 hours. The MSC cells and supernatants will be obtained from Case Western Reserve University Cancer Center (see IRB). Dr. Caplan's laboratory will be validating the in vitro efficacy of the stem cells themselves using the ceramic cube model. Currently this is the only accepted model for evaluating the overall “health” of MSC cell preparations.

TABLE 1
Pathogen of Interest and Antibiotic of Choice

| Pathogen | Antibiotic | In vitro Dosing |
|---|---|---|
| Pseudomonas aeruginosa | Gentamicin, Ceftazidine, | 0.25 mg/ml, |
| | Tobramicin | 1.0 mg/ml, |
| | | 2.0 mg/ml |
| Staphylococcus aureus | Penicillin, Cephalosporins | |
| Streptococcus pneumoniae | Ceftriazone, Ciprofloxacin | |

The growth of the bacteria will be measured by counting colony forming units (CFUs), turbidity assay (optical density), growth kinetics and viability. The CFUs tell us how the bacteria can recover and grow from the MSC product treatment. The CFUs will be counted, 24 hours after the bacteria-MSC combinations are streaked onto either tryptic soy agarose plates (for Pseudomonas aeruginosa, Staphylococcs aureus) and blood agar plates (for Streptococcus pneumonia). The counting is done manually using a colony counting pen. The turbidity assay tells us the immediate impact of the MSC products on bacteria numbers. Aliquotes of bacteria are measured for the ability to diffract light at 400 nm (light visibility) resulting in a designated optical density. The growth kinetics will measure how the MSC products impact the ability of the bacteria to grow over time determining the sustainability of the treatment. ATP is a product of cell growth, and when combined with the proprietary luminescence reagent, can be used to measure growth kinetics over time. The growth kinetics will be measured by evaluating the generation of bacterial ATP by luminescence using BacTiter-Glo™ Microbial Cell Viability Assay (Promega, Madison Wis.). This is cost-effective luminescence assay which can measure several samples at the same time, following all of the time points and conditions of measurements. The viability determines whether the impact of the MSC products is on bacteria death or slowing growth, both important in the mechanisms of bacteremia and sepsis. The viability of the bacteria is determined by flow cytometry using LIVE/DEAD® BacLight™ Bacterial Viability and Counting Kit (Life Technologies, Grand Island N.Y.). The assay uses two dyes: propidium iodine and SYTO 9 to stain the bacteria, with both stains being excited by 488 nm spectral line. With the appropriate mixture of the SYTO 9 and propidium iodide stains, bacteria with intact cell membranes fluoresce green whereas bacteria with damaged membranes fluoresce red. The cell type and the gram character influence the amount of red-fluorescence staining exhibited by dead bacterial. Our studies will evaluate the presence of live versus dead bacteria at each time-point post-treatment with the MSC products. These studies are justified by the preliminary data shown in FIG. 12. MSC-supernatants decrease Pseudomonas aeruginosa (12A), Staphylococcus aureus (12B) and Streptococcus pneumonia (12C) CFUs. Further, combined MSC products and antibiotics resulted in even greater suppression of bacteria CFUs regardless of the bacteria. In FIG. 12C, is the growth kinetics of Pseudomonas aeruginosa in response to MSC-products, showing that the decreased CFUs in FIG. 12A is due to slower growth of the bacteria. In the final part of this aim, aliquots from each of the supernatant preparations will be analyzed to identify potential antimicrobial peptides. The statistics will be done with 2-sided t-tests and a significance of P≦0.05 with MSC donors serving as replicates. These exploratory studies can detect anticipated differences with 90% power.

In the second part of these studies we will begin to bridge the bench-side research to clinical application using sputum and pulmonary aspirates from pediatric patients. When children are admitted to the hospital with pneumonia or develop pneumonia as a consequence of hospitalization one component of the clinical diagnostics is evaluating the sputum or tracheal aspirates for type of bacteria and their sensitivity to antibiotics. These are diagnostic laboratory specimens used to identify antibiotic sensitivity for treatment. Once the antibiotic sensitivity is established the samples are disposed but are available as non-human samples requiring no patient identifiers, just the disease context of the sample. We will begin with cystic fibrosis sputum since it is an easily accessible clinical sample, and often colonized by bacteria including Pseudomonas aeruginosa, Staphyloccus aureus and Streptococcus pneumonia, Hemophilus Influenza and others. We will evaluate 20 patient samples for the ability of the MSCs and their secreted products to treat the pathogens present on the microbiology sample using the conditions identified in Aim 1, pursuing CFUs, turbidity, growth kinetics and viability (FIG. 14). These studies are important because real infections in children may involve more than one pathogen which might interfere with the overall effectiveness of the MSCs and their products. In addition, we can also take advantage of the antibiotic sensitivity results, evaluating the MSC products with and without the clinically determined antibiotic. The idea is that the MSC products will enhance the sensitivity of the bacteria to first line antibiotic treatment, saving more sophisticated antibiotics for those cases that absolutely require application. The concept of these studies is significant since this may prevent unnecessary antibiotic usage, thus helping to limit the development of resistant strains. These studies will be the foundation for the development of an investigator initiated clinical trial using MSC products as additive therapy in scenarios of severe pneumonia and sepsis.

### Aim 2. To Determine the Efficacy, Potency and Sustainability of the MSCs Products In Vivo Using Murine Models of Chronic Pneumonia and Sepsis

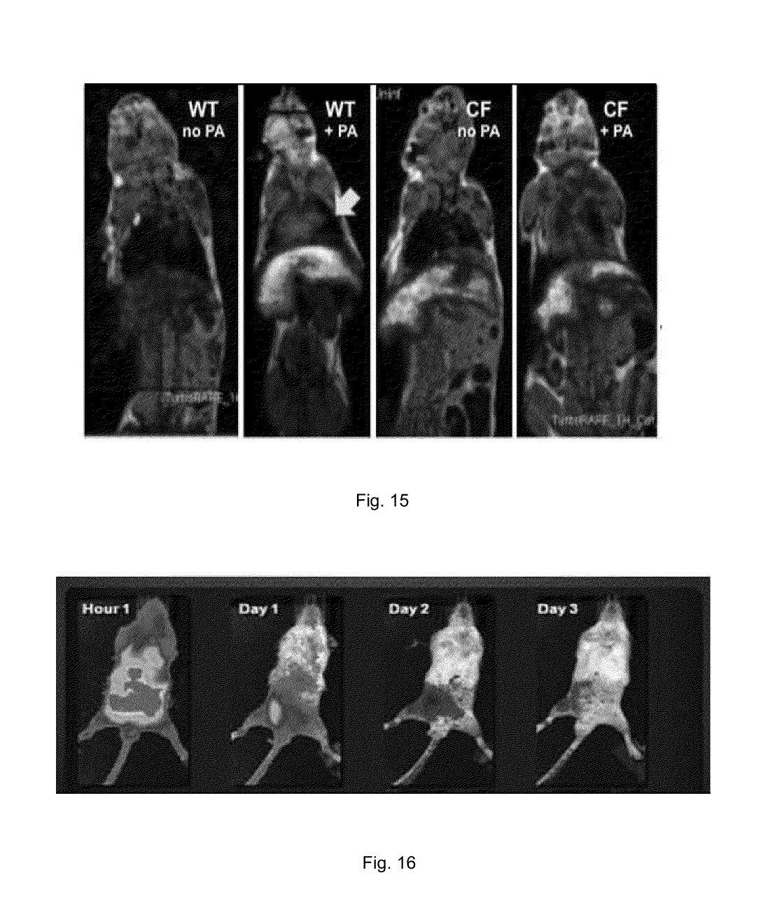

There are a variety of bacteria that can be used for investigations into antimicrobial benefits of MSCs in vivo. We will focus on a gram positive (Staphlococcus aureus) and gram negative (Pseudomonas aeruginosa) pathogens because they are highly associated with nosocomial pneumonia and sepsis in children. We will also take advantage of our established model of chronic Pseudomonas aeruginosa pneumonia model in mice that mimic the chloride transporter defect, cystic fibrosis transmembrane regulator (CFTR). These animals (Cftr^{tm1Unc}) are highly susceptible to chronic infection and inflammation. In these experiments we will use both C57BL/6J and Cftr^{tm1Unc} models. The chronic pneumonia model is established, by impregnating agarose beads with bacteria 10⁶ viable bacterial-agarose bead preparation will be administered into the lungs of mice using a fine gauge needle, by surgically inserting the needle gently into the trachea and placing the bacteria-agarose bead preparation into the left lobe of the lung. 1-day after the start of infection, animals will be given 10⁶ MSCs or 100 μg/μl MSC products intranasally. Pneumonia induced sepsis is established by anesthetizing the animals with intranasal administration 10⁷ viable bacteria. The majority of animals given this bolus of bacteria die due to the infection within 7 days of administration. For the sepsis model, MSCs or their products will be given 3 hour post-infection. Animals will be imaged at day 2, 4, 6 and euthanized on day 7 for lung pathology or BAL pathophysiology with differentials and cytokines. In addition one set of animals will be treated with gentamicin at a dose of 2 mg/kg, which is sub-optimal for infection resolution in vivo. The mice will be imaged for MSC localization using Luciferase and Red Fluorescent Protein labeling of the MSCs in collaboration with Dr. Christopher Flask (see letter of support). The inflammatory response in the infected animals will be followed using an MRI-sensitive phagocyte marker (¹⁹F), in collaboration with Christopher Flask, Ph.D., a co-investigator from the Department of Radiology (see letter of support). ¹⁹F is a nanoparticle that is rapidly scavenged by phagocytic cells and can be readily imaged by MRI (22-24). Our preliminary data in FIG. 15 shows the diffuse recruitment of phagocytic cells in the lungs of WT mice 48 hours after infection with agarose beads impregnated with Pseudomonas aeruginosa (panel B), compared to WT animals not infected (panel A). These WT mice also had increased ¹⁹F nanobead up-take in the liver. The diffuse recruitment of phagocytic cells was not observed in the CF lung at 48 hours post-Pseudomonas infection (panel D), but evidence of immune activation is observed in the liver even without infection (panel C). FIG. 16 shows and example of labeled MSCs in a mouse with a leg injury and the change between global administration of MSCs (1 hour) and localization to the site of injury (day 3). In the studies outlined in this proposal, the animals will be followed for 7 days and imaged at day 2, 4 and 6. All animals will have daily clinical scores, weight changes and temperature (see Table II, Methods for criteria of clinical score—). At 7 days post-infection, animals are euthanized and lungs will be scored for gross lung pathology followed by bronchoalveolar lavage (BAL) which is used to wash out the lungs.

TABLE 2
Clinical and Lung Histological Scoring Criteria

| Score | Clinical Score | Gross Lung Pathology |
|---|---|---|
| 0 | Healthy appearance and activity | Within normal limits |
| 1 | Scruffy appearance | Darker red |
| 2 | Scruffy and dehydrated | Few nodules |
| 3 | Scruffy, dehydrated, decreased | Several nodules, <25% |
| | activity | consolidation |
| 4 | Scruffy, dehydrated, minimal | Numerous nodules 25-50% |
| | activity | consolidation |
| 5 | Moribund or dead | Numerous nodules >50% |
| | | consolidation |

*Clinical scores and gross lung pathology were evaluated by two different individuals, unaware of their group designation. Validation of clinical score and gross lung pathology Criteria is extensively described in van Heeckeren, AM 2002 and van Heeckeren, AM 2000.

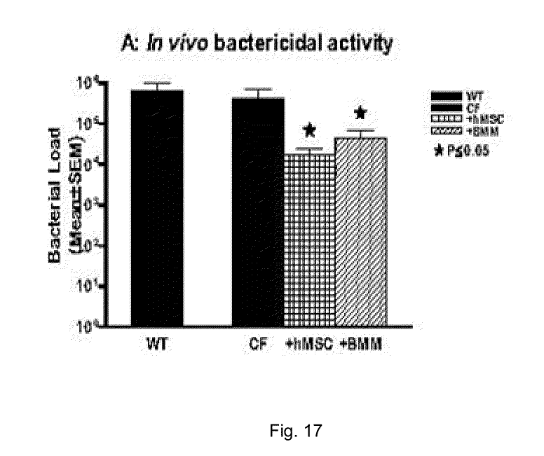

The lung exudates will be evaluated for total cell count, differential (type of inflammatory cells) and bacteriology which will define the level of infection as well as the animal's inflammatory response. The remaining lung tissue is evaluated for bacteriology. The bacteriology will consist of investigating the remaining CFUs relative to initial inoculums and the viability of the bacteria as described in Aim 1. Serum and BAL fluid will be evaluated for cytokines associated with infection and the inflammatory response including: TNFα, IL-1β, IL-6, IL-10 and MIP-1α using Luminex multiplex technology. The Principle Investigator is director of Bioanalyte Core and the Cystic Fibrosis Lung Infection and Inflammation Modeling Core with extensive experience in both the animal models and Luminex technology. FIG. 17, shows our preliminary data using the Pseudomonas aeruginosa pneumonia model and administration of MSC cells. In these studies we administered 106 MSC-cells 1 day after initiation of infection. Although our model demonstrates sustained pulmonary infection with Pseudomonas aeruginosa, the MSC-cells decreased the bacteria load, trajectory of weight loss, and clinical scores by day 10, all consistent with improved outcomes in the model. These studies are designed to investigate the clinical potential of using the MSC products in scenarios of chronic lung infection and the impact on antibiotic therapy.

### Aim 3. To Develop a Drug Delivery System which Will Improve the Duration and Sustainability of the MSCs and their Products In Vitro and In Vivo

Our preliminary data suggests that the antimicrobial activity in the supernatant works only for a limited period of time. Using the cell has the potential to provide a sustainable resource of the antimicrobial activity, however keeping them specifically associated with the site of infection may be an issue since they have the potential of traveling to sites of injury beyond that of the infection. In this aim we will work closely with our colleagues in Biomedical Engineering (Horst von Recum, see letter of support and biosketch) to develop a drug delivery system that will either deliver the soluble antimicrobial supernatant or enclose MSCs for delivery to the infected site. In this final aim, we will create drug delivery systems in which MSC small products will be encapsulated in polymers. We will test the antimicrobial activity of the MSCs in vitro first against Pseudomonas aeruginosa, Streptococcus pneumonia and Staphylococcus aureus.

### Expected Results and Alternative Approaches

The preliminary data already suggests that these studies will be successful at showing the overall benefit of MSC-products for treating pediatric infections. With each aim there is a significant impact and potential for clinical application. For Aim 1, the data will support treating external infections and enhancing antibiotic efficiency with a focus on both simple and complex infections. For Aim 2, MSCs and their products will be used to treat pulmonary infections using a model which mimics chronic and destructive Pseudomonas aeruginosa or Staphylococcus aureus pneumonia. The issue that will be closely monitored include the MSCs, the MSC derived products and the variability inherent in the animal studies. We will monitor the response of the MSCs using an in vitro ceramic cube assay developed by the inventors. We can also optimize the production of these MSC products by stimulating the cells with IFNγ (100 U/ml) and comparing the antimicrobial activity to cells that have not been stimulated. In vivo applications will take advantage of our extensive experience with the in vivo model. In minimizing the variability in the in vivo models will include doing all of the groups at the same time: infected mice, infected mice with MSC products, infected mice with antibiotic, infected mice with both antibiotic and MSC products.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may be different from the actual dates, which may need to be independently confirmed.

Having described the invention, the following is claimed:

1. A method of treating a microbial infection in a subject comprising administering to the subject a therapeutically effective amount of MSCs, wherein the therapeutically effective amount is the amount effective to inhibit microbial growth and suppress microbial infection associated inflammation in the subject.

2. The method of claim 1, wherein the MSCs are provided in a pharmaceutical composition with a pharmaceutically acceptable carrier and administered to the subject via intramuscular, inhalation, intravenous or retro-orbital administration.

3. The method of claim 1, wherein the MSCs are allogeneic or autologous MSCs.

4. The method of claim 1, the MSCs comprising preconditioned MSCs, wherein the preconditioned MSCs are cultured in a microbial infection associated disease setting prior to administration of the MSCs to the subject.

5. The method of claim 4, the disease setting comprising one or more inflammatory cytokines related to a microbial infection.

7. The method of claim 5, the inflammatory cytokines selected from the group consisting of TNFα, IFNγ, IL-1β, IL-6, IL-10, IL-17 and MIP-1α.

8. The method of claim 1, wherein the microbial infection is a respiratory microbial infection associated with cystic fibrosis, the microbial infection selected from the group consisting of Pseudomonas aeruginosa, Staphylococcus aureus, and Streptococcus pneumoniae microbial infection.

9. A method of treating a microbial infection in a subject comprising administering to the subject a therapeutically effective amount of MSCs and an antimicrobial agent, wherein the MSCs potentiate the therapeutic activity of the antimicrobial agent, and wherein the therapeutically effective amount is the amount effective to inhibit microbial growth and suppress microbial infection associated inflammation in the subject.

10. The method of claim 9, the amount of the antimicrobial agent comprising a subtherapeutic amount of the antimicrobial agent.

11. The method of claim 9, wherein the MSCs and antimicrobial agent are provided in a pharmaceutical composition with a pharmaceutically acceptable carrier and administered to the subject via intramuscular, inhalation, intravenous or retro-orbital administration.

12. The method of claim 9, the antimicrobial agent selected from the group consisting of an antibiotic, antiviral or antifungal agent.

13. The method of claim 12, the antimicrobial agent comprising a β-lactam antibiotic agent.

14. The method of claim 9, wherein the MSCs are allogeneic or autologous MSCs.

15. The method of claim 9, the MSCs comprising preconditioned MSCs, wherein the preconditioned MSCs are cultured in a microbial infection associated disease setting prior to administration of the MSCs to the subject.

16. The method of claim 15, the disease setting comprising one or more inflammatory cytokines related to a microbial infection.

17. The method of claim 16, the inflammatory cytokines selected from the group consisting of TNFα, IFNγ, IL-1β, IL-6, IL-10, IL-17 and MIP-1α.

18. The method of claim 9, wherein the microbial infection is a respiratory microbial infection associated with cystic fibrosis, the microbial infection selected from the group consisting of Pseudomonas aeruginosa, Staphylococcus aureus, and Streptococcus pneumoniae microbial infection.

19. A method of treating a respiratory microbial infection associated with cystic fibrosis in a subject comprising:
administering to the subject a therapeutically effective amount of MSCs and an antimicrobial agent, wherein the MSCs potentiate the therapeutic activity of the antimicrobial agent, and wherein the therapeutically effective amount is the amount effective to inhibit microbial growth and suppress respiratory microbial infection associated inflammation in the subject.

20. The method of claim 19, the amount of the antimicrobial agent comprising a subtherapeutic amount of the antimicrobial agent.
